(19) United States
(12) Patent Application Publication
(10) Pub. No.: US 2010/0248310 A1
(43) Pub. Date: Sep. 30, 2010

(54) METHOD OF DNA AMPLIFICATION

(75) Inventor: Alexander Alan Morley, Glenelg (AU)

Correspondence Address: KNOBBE MARTENS OLSON & BEAR LLP, 2040 MAIN STREET, FOURTEENTH FLOOR, IRVINE, CA, 92614, US

(73) Assignee: Monoquant Pty Ltd., Adelaide, South Australia (AU)

(21) Appl. No.: 12/299,007

(22) PCT Filed: Oct. 2, 2008
(86) PCT No.: PCT/AU2008/001453

§ 371 Date: May 20, 2010

Related U.S. Application Data
(60) Provisional application No. 60/981,761, filed on Oct. 22, 2007.

(51) Int. Cl. C12P 19/34

(52) U.S. Cl. 435/91.2; 435/91.5

(57) ABSTRACT

The present invention relates generally to a method of amplifying a nucleic acid region of interest and, more particularly, to a method of amplifying a nucleic acid region of interest using a PCR method designed to minimise the generation of amplicons from primers which have bound to nucleic acid regions other than the specific region of interest. The method of the present invention is based on the determination that by rendering inefficient the functionality of either the forward primer or the reverse primer, the rate of amplification of irrelevant nucleic acid regions can be reduced relative to amplification of the region of interest. The provision of a selective means of amplifying a nucleic acid region of interest is useful in a range of applications including, but not limited to, the diagnosis and/or monitoring of disease conditions which are characterised by specific gene sequences, the characterisation or analysis of gene regions of interest, the identification or characterisation of DNA breakpoint regions and the isolation of gene sequences of interest where only the nucleotide sequence at one end of the gene sequence of interest is known.

### FIELD OF THE INVENTION

The present invention relates generally to a method of amplifying a nucleic acid region of interest and, more particularly, to a method of amplifying a nucleic acid region of interest using a PCR method designed to minimise the generation of amplicons from primers which have bound to nucleic acid regions other than the specific region of interest. The method of the present invention is based on the determination that by rendering inefficient the functionality of either the forward primer or the reverse primer, the rate of amplification of irrelevant nucleic acid regions can be reduced relative to amplification of the region of interest. The provision of a selective means of amplifying a nucleic acid region of interest is useful in a range of applications including, but not limited to, the diagnosis and/or monitoring of disease conditions which are characterised by specific gene sequences, the characterisation or analysis of gene regions of interest, the identification or characterisation of DNA breakpoint regions and the isolation of gene sequences of interest where only the nucleotide sequence at one end of the gene sequence of interest is known.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Bibliographic details of the publications referred to by author in this specification are collected alphabetically at the end of the description.

The polymerase chain reaction (PCR) is a technique which is utilised to amplify specific regions of a DNA strand. This may be a single gene, just a part of a gene or a non-coding sequence. Most PCR methods typically amplify DNA fragments of up to 10 kilo base pairs (kb), although some techniques allow for amplification of fragments up to 40 kb in size (Cheng et al., 1994, Proc Natl Acad Sci. 91:5695-5699).

PCR, as currently practiced, requires several basic components (Sambrook and Russel, 2001, Molecular Cloning: A Laboratory Manual, 3^{rd} Ed.). These components are:
a DNA template which contains the region of the DNA fragment to be amplified;
primers, which are complementary to the DNA regions at the 5′ and 3′ ends of the DNA region that is to be amplified;
a DNA polymerase (e.g. Taq polymerase or another thermostable DNA polymerase with a temperature optimum at around 70° C.), used to synthesize a DNA copy of the region to be amplified; and
Deoxynucleotide triphosphates (dNTPs) from which the DNA polymerase builds the new DNA.

PCR is carried out in small reaction tubes (0.2-0.5 ml volumes), containing a reaction volume typically of 15-100 μl, which are inserted into a thermal cycler. This machine heats and cools the reaction tubes within it to the precise temperature required for each step of the reaction. Most thermal cyclers comprise heated lids to prevent condensation on the inside of the reaction tube caps. Alternatively, a layer of oil may be placed on the reaction mixture to prevent evaporation.

Accordingly, PCR is a method that allows exponential amplification of DNA sequences within a longer DNA molecule. The reaction involves a number of cycles of amplification, and in each cycle the template for each molecular reaction is either a strand of genomic DNA or a strand of DNA synthesised in a preceding cycle. Each PCR cycle involves the following steps
denaturation by heat to separate the 2 strands of double-stranded DNA molecules
hybridisation of the upstream and downstream primers to their complementary sequences
extension of the primers by the DNA polymerase to produce a complementary copy of the template sequence

Typically the PCR reagents and conditions are chosen so that denaturation, hybridisation and extension occur at close to maximum efficiency and as a result the amount of the desired sequence increases with each cycle by a factor of close to 2. Substantial amplification occurs by the end of the PCR eg a 30 cycle PCR will result in amplification of the original template by a factor of almost 2³⁰ (1,000,000,000). This degree of amplification facilitates detection and analysis of the amplified product

Other nucleic acid amplification techniques, such as the Ligase Chain Reaction (LCR) or the Nucleic Acid Sequence Based Reaction (NASBA), are also used to amplify a desired sequence in DNA. The reaction strategies differ from that of the PCR but they also use primers that hybridise to the 2 ends of the target sequence and again, the reaction is typically performed to ensure that each step, including hybridisation, occurs at or close to maximum efficiency. Although the ensuing discussion is largely directed towards PCR, the concepts equally apply to other amplification techniques.

After a number of cycles of amplification, the PCR product can be analyzed in various ways, most commonly by gel electrophoresis. In its simplest form this method of analysis is semi-quantitative in its simplest form. The amount of product is not closely related to the amount of input DNA, thereby making this type of PCR a qualitative tool for detecting the presence or absence of a particular DNA.

In order to measure messenger RNA (mRNA), the method uses reverse transcriptase to initially convert mRNA into complementary DNA (cDNA) which is then amplified by PCR and analyzed by agarose gel electrophoresis. In many cases this method has been used to measure the levels of a particular mRNA under different conditions. However, this method is actually even less quantitative than the PCR of DNA because of the extra reverse transcriptase step.

In order to provide quantitation capabilities, real-time PCR was developed. This procedure follows the general pattern of PCR, but the amplified DNA is quantified during each cycle. Two common methods of quantification are the use of fluorescent dyes that intercalate with double-stranded DNA and modified DNA oligonucleotide primers or probes the fluorescence of which changes during one of the steps of the PCR. Frequently, real-time polymerase chain reaction is combined with reverse transcriptase polymerase chain reaction to quantify low abundance messenger RNA (mRNA), enabling a researcher to quantify relative gene expression at a particular time or in a particular cell or tissue type.

### (i) Real-Time PCR Using Dyes Binding to Double-Stranded DNA

A DNA-binding dye binds to all double-stranded (ds)DNA in a PCR reaction, causing increased fluorescence of the dye. An increase in DNA product during PCR therefore leads to an increase in fluorescence intensity which is measured at each cycle, thus allowing DNA concentrations to be quantified. Like other real-time PCR methods, the values obtained do not have absolute units associated with them (i.e. mRNA copies/cell). Accordingly, a comparison of a measured DNA/RNA sample to a standard dilution will only give a fraction or ratio of the sample relative to the standard, allowing only relative comparisons between different tissues or experimental conditions. To ensure accuracy in the quantification, it is usually necessary to normalize expression of a target gene to a stably expressed gene. This can correct for possible differences in RNA quantity or quality across experimental samples.

### (ii) Fluorescent Reporter Sequence Methods

A number of different methods using fluorescent reporter primers or probes have been developed and they tend to be more accurate and reliable than use of DNA binding dyes. They use one or more DNA primers or probes to quantify only the DNA to which the primer or probe hybridises. Use of a reporter probe significantly increases specificity and may allow quantification even in the presence of some non-specific DNA amplification. Use of sequence-specific primers or probes allows for multiplexing—assaying for several different amplified products in the same reaction by using specific sequences or probes with different-coloured labels, provided that all targets are amplified with similar efficiency.

In terms of quantitation, relative concentrations of DNA present during the exponential phase of the reaction are determined by plotting fluorescence against cycle number on a logarithmic scale. A threshold for increase of fluorescence above background or decrease below background (depending on the precise method) is determined. The cycle at which the fluorescence from a sample crosses the threshold is called the cycle threshold, Cₜ. Since the quantity of DNA doubles every cycle during the exponential phase, relative amounts of DNA can be calculated, e.g. a sample whose Cₜ is 3 cycles earlier than another's has 2³=8 times more template (assuming that the amount of amplified DNA doubles with each cycle).

Amounts of DNA are then determined by comparing the results to a standard curve produced by serial dilutions (e.g. undiluted, 1:4, 1:16, 1:64) of a known amount of DNA.

However, one of the limitations of PCR relates to the fact that primers can, in some situations, bind to more than one region of a DNA sample, thereby potentially leading to the generation of amplified sequences which are unrelated to the DNA sequence of interest. Binding to multiple regions may occur in a number of situations which include but are not limited to
1. Non-specific binding of the primer owing to, for example, the primer being very short or degenerate or of a design which favours non-specific binding or subject to PCR conditions which favour non-specific binding.
2. The sequence to which a primer binds naturally occurs in many regions of the genome. Sequences belonging to families such as alu or line are widely dispersed and, although individual sequences may vary slightly, their homology is such that a primer binding to one member of a family is likely to bind to numerous other members.
3. The sequence to which a primer binds has been introduced into many regions of the genome. This may occur, for example, if the DNA has been digested by a restriction enzyme and a common sequence has been ligated at the sites of digestion.
4. The presence of multiple primers in the reaction. By chance, one primer may bind so as to act as a forward primer and another may bind so as to act as a reverse primer. The probability of occurrence of this phenomenon will increase as the number of primers increases.

As a consequence of one of these situations, it may be the case that one primer binds so as to act as a forward primer and the same or another primer binds so as to act as a reverse primer. If the binding sites are sufficiently close, non-specific amplification may occur. If one considers that the capacity of PCR to amplify over one billion fold also increases the possibility of amplifying the wrong DNA sequence over one billion times, the importance of minimising this possibility becomes clear.

Accordingly, there is an ongoing interest and need to develop means of overcoming this problem. The usual approach is nested PCR. In this method two pairs of PCR primers are used for a single locus. The first pair amplifies the locus as seen in any PCR experiment. The second pair of primers (nested primers) bind within the first PCR product and produce a second PCR product that will be shorter than the first one. The logic behind this strategy is that if an unwanted locus was also amplified, the probability is very low that it would also be amplified a second time by a second pair of primers. However nested PCR is only of value if some or all of the DNA sequence internal to the first pair of primers is known so that 1 or more internal primers which provide additional specificity can be synthesised.

In work leading up to the present invention, another method of minimising unwanted amplification events has been developed. Specifically, it has been determined that if one of the two PCR primers (e.g. primer 2) is designed or used such that it hybridises inefficiently and comprises a nucleic acid tag which can itself lead to a binding site for a third (tag) primer, a “bottleneck” in amplification will occur. The bottleneck occurs because efficient amplification of the desired product will only commence after primer 2 has hybridised and extended, and this process is inefficient. In subsequent cycles, exponential amplification initiated from the templates so produced is efficient, being mediated by efficient hybridisation and extension of primer 1 in one direction and efficient hybridisation and extension of the tag primer in the other direction. However, for undesired amplicons generated from sequences for which primer 2 can act as both a forward and a reverse primer, the bottleneck during the PCR will be much more severe. For such sequences, efficient amplification by the tag primer would only occur after 2 sequential and inefficient hybridisations of primer 2 have occurred, one in the forward and one in the reverse direction. Thus, by deliberately ensuring that hybridisation of one primer in the PCR is rendered inefficient, one can select against amplification of sequences for which that primer acts as both a forward and reverse primer, and favour amplification of sequences which are amplified from one end by that primer and from the other end by an efficiently hybridising primer.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a particular integer or group of integers has originated from the species specified, but has not necessarily been obtained directly from the specified source. Further, as used herein the singular forms of “a”, “and” and “the” include plural referents unless the context clearly dictates otherwise.

Unless otherwise defined, all technical and scientific terms used herein have the same meaning as commonly understood by one of ordinary skill in the art to which this invention belongs.

One aspect of the present invention provides a method of amplifying a nucleic acid region of interest, said method comprising:
(i) contacting a nucleic acid sample with:
   (a) one or more forward primers directed to said region of interest; and
   (b) one or more reverse primers directed to said region of interest
   wherein the primers of either group (a) or group (b) are functionally inefficient and are operably linked at their 5′ end to an oligonucleotide tag;
(ii) amplifying the nucleic acid sample of step (i) through at least two cycles of amplification;
(iii) contacting the amplified nucleic acid of step (ii) with a primer which is directed to part or all of the sequence which is complementary to that of the oligonucleotide tag of step (i); and
(iv) amplifying the nucleic acid sample of step (iii).

Another aspect of the present invention provides a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) are functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

In yet another aspect the present invention provides a method of amplifying a gene or gene fragment of interest, said method comprising:
(i) contacting a DNA sample with:
   (a) one or more forward primers directed to said gene or gene fragment of interest; and
   (b) one or more reverse primers directed to said gene or gene fragment of interest
   wherein the primers of either group (a) or group (b) are functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

In still another aspect, there is provided a method of amplifying a chromosomal gene translocation breakpoint region of interest, said method comprising:
(i) contacting a DNA sample with:
   (a) one or more forward primers directed to said chromosomal gene translocation breakpoint region of interest; and
   (b) one or more reverse primers directed to said chromosomal gene translocation breakpoint region of interest
   wherein the primers of either group (a) or group (b) are functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

In yet still another aspect, there is provided a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) are functionally inefficient and are operably linked at their 5′ end to an oligonucleotide tag and wherein said functionally inefficient primer group is the primer group which has the potential to hybridise promiscuously to non-target DNA regions;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

In still yet another aspect, there is provided a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) hybridise inefficiently and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

A further aspect of the present invention provides a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) hybridise inefficiently and are operably linked at their 5′ end to an oligonucleotide tag;
(ii) amplifying the DNA sample of step (i) through at least two cycles of PCR;
(iii) contacting the amplified DNA of step (ii) with a primer which is directed to part or all of the sequence which is complementary to that of the oligonucleotide tag of step (i); and
(iv) amplifying the DNA sample of step (iii) by PCR.

Yet another aspect of the present invention is directed to a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) hybridise inefficiently and are both functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i);
(iv) amplifying the DNA sample of step (iii); and
(v) isolating and/or analysing said amplified DNA.

### BRIEF DESCRIPTION OF THE DRAWINGS

FIG. 1 is a schematic representation depicting, for amplification of the sequence of interest, the 2 events in 2 (not necessarily successive) PCR cycles which must occur before efficient amplification occurs. The first is when the inefficient primer 2 hybridises and extends to form an antisense strand, the second is when the efficient primer 1 hybridises to the resultant antisense strand and extends to form a sense strand containing the binding sequence for the tag primer. Efficient amplification in subsequent cycles then occurs primed by primer 1 and the tag primer.

FIG. 2 is a schematic representation of negative selection during bottleneck PCR. In classical PCR there is efficient hybridisation and extension at both upstream and downstream primer sites. In Bottleneck PCR there is a bottleneck owing to inefficient hybridisation at either one or both of the primer-binding sites. This results in negative selection for targets for which there is a bottleneck at each end as compared to those for which there is a bottleneck at only one end.

FIG. 3 is a schematic representation of the experimental design for enrichment of BCR-ABL breakpoint sequence using Bottleneck PCR. The second, third and fourth PCR rounds were bottleneck PCRs, as the hybrid-tag downstream primers were present at a low concentration, resulting in inefficient hybridisation and extension. There was also some incidental bottleneck effect in the first round owing to the low concentration of each downstream ABL primer. In a typical experiment, the first 3 rounds would each involve 20 PCR cycles and the fourth final round would involve 20-35 cycles.

FIG. 4 is a schematic representation of the experimental design for enrichment of a desired product using bottleneck PCR. The primary PCR involves a forward gene-specific primer (F1) and either a degenerate (universal) reverse primer, if gene walking is being performed, or a pool of reverse primers, if amplification across a translocation breakpoint is being performed. The primary PCR can be designed as a bottleneck PCR by rendering the reverse primer(s) inefficient and, when a pool of reverse primers is used, actually performs as a bottleneck PCR owing to the low concentration of each reverse primer. The second, and third PCR rounds are definitive bottleneck PCRs. They involve the same or a nested forward primer and a low concentration of the hybrid-tag reverse primer, which results in inefficient hybridisation and extension of this primer. In a typical experiment, the first 2 or 3 rounds would each involve 20 PCR cycles and the final round would involve 20-35 cycles.

FIG. 5 is a graphical representation of the enrichment of specific product by selection against non-specific products. The specific product was the BCR-ABL translocation sequence and the non specific products were anonymous genomic sequences to which one or more of the ABL primers hybridised at each end resulting in amplification. DNA from a patient with the BCR-ABL translocation was amplified in round 1 with an upstream BCR primer and 282 downstream tagged ABL primers. Three successive rounds of bottleneck PCR were then performed, each using an upstream BCR primer and a downstream linking hybrid tag, tagᵢ₊₁ primer and a tagᵢ₊₁ primer. Amplification during each round was quantified, using sequence-specific primers for the BCR ABL translocation and the tag primer for the non-specific products. As shown by the Ct values for the 2 products, non-specific product greatly predominates in the first round but its relative amount decreases with each round so that specific product predominates in rounds 3 and 4. The degree of enrichment observed between rounds 2 and 1 and between rounds 3 and 2 suggests that the tag-tag primers were acting at approximately 1% of maximum efficiency.

FIG. 6 is an image of the selective effect of bottleneck PCR in facilitating isolation of translocation breakpoint regions. Lanes 2, 5 and 17 show results of amplification with DNA from 3 patients with chronic myeloid leukaemia, lane N shows results with DNA from a normal control and lane MQ shows results from a water control. In the first cycle of PCR amplification, all samples were amplified with 1 upstream primer directed against BCR and 282 primers directed against the ABL gene. For one set of amplifications, the first round was continued for 45 cycles (1^{st} round), whereas for the other set of amplifications the PCR was stopped after 20 cycles and a 1/100 aliquot was used to perform 3 further rounds of Bottleneck PCR (4^{th} cycle). The 1^{st} round results show a broad smear of heterogeneous products which have been amplified owing to priming at both ends by the ABL primers. The 4^{th} round products are much less complex and homogeneous bands can be seen. There is one non-specific band seen in all samples, and in patients 2 and 5 the band due to amplification of the BCR ABL translocation can be seen (arrows).

FIG. 7 is another image of the selective effect of bottleneck PCR. Lanes 6, 16 and 23 show results of amplification with DNA from 3 patients with chronic myeloid leukaemia, lane N shows results with DNA from a normal control and lane MQ shows results from a water control. In the first round of PCR amplification, all samples were amplified with 1 upstream primer directed against BCR and 282 primers directed against the ABL gene; in the second round, all samples were amplified with 1 nested upstream primer directed against BCR and 282 nested primers directed against the ABL gene. When Bottleneck PCR was not performed, the second round was continued for 45 cycles, whereas, when it was performed, the second round PCR was stopped after 20 cycles and a 1/100 aliquot was used to perform 2 further rounds of bottleneck PCR. This resulted in preferential amplification of the BCR-ABL sequence in the 3 patients (indicated by arrows).

FIG. 8 is a graphical representation depicting a model of amplification produced in a classical PCR or in a single-round bottleneck PCR in which either one primer or both primers are inefficient. The model was produced using the Excel spreadsheet. In the example shown, the starting target for each PCR is one DNA duplex, comprising a sense and an antisense strand, and the probability of hybridising and extending during each cycle is 1 for the efficient primer(s) and 0.001 for the inefficient primer(s). The bottleneck before exponential amplification predominates is evident. After any given number of PCR cycles, the target amplified by one efficient and one inefficient primer has increased approximately 1000-fold compared to the target amplified by the two inefficient primers.

FIG. 9 shows images from an experiment using bottleneck PCR to isolate the PML-RARα translocation breakpoint from a patient with acute promyelocytic leukemia. a) The patient DNA was amplified using multiple RARα primers and a single PML primer and then 2 rounds of bottleneck PCR were performed. The Figure shows the amplified DNA electrophoresed on a 2% agarose gel. P is patient DNA, N is the normal DNA and W is the water control. The DNA ladder shows bands 700 bp to 100 bp descending from top to bottom. The patient band can be seen at approximately 500 bp. b) To confirm that the breakpoint had been isolated, the breakpoint sequence was used to design RARα and PML primers spanning the breakpoint, and the patient DNA was amplified for one round using these primers. The Figure shows the amplified DNA electrophoresed on a 2% agarose gel. P is the patient DNA, N is the normal DNA and W is the water control. The DNA ladder shows bands 700 bp to 100 bp descending from top to bottom. The confirmatory patient band can be seen at approximately 150 bp. c) The sequence chromatogram obtained from the amplified band shown on the gel in section a of the figure. The breakpoint between the PML and RARα is shown.

FIG. 10 shows images illustrating the use of bottleneck PCR for gene walking, using a “universal” degenerate primer, along the Myocillin gene using 4 different degenerate primers (lanes 1-4), which were identical except that their fifth most 3′ base was either A, G, C or T. The first round is the primary PCR and the second and third rounds are bottleneck PCRs. The decrease in complexity with serial bottleneck PCRs is apparent. Sequencing of the predominant band in each of the four amplifications showed the expected Myocillin gene sequence Lane M is a DNA ladder with products from 700-100 by at 100 bp intervals, W is the water control with no DNA.

FIG. 11 is a graphical illustration of progressive selection against non-specific amplification products by bottleneck PCR. The experiment studied walking along the APC gene. The primary PCR used 20 cycles with one gene-specific and one degenerate primer and was followed by 3 rounds of bottleneck PCR each of 20 cycles. Amplification products were sampled every 5 cycles and specific and non-specific products were assayed in a “read-out” PCR. A decreasing Ct indicates increasing product. Specific product starts to predominate after 1 bottleneck PCR.

FIG. 12 shows images of APC gene sequences from a three round genewalking experiment. A primary PCR using an APC gene-specific primer and a degenerate reverse primer was followed by 2 rounds of bottleneck PCR and the amplification product was sequenced directly, without electrophoresis. Three APC sequences in the forward direction, using different sequencing primers, are shown. The sequencing primer used to obtain A was the same as that used in the third PCR round. The sequencing primers used to obtain B and C were 732 and 1302 bps respectively from the most 5′ prime primer. The total length of readable sequence was approximately 1.5 kb.

### DETAILED DESCRIPTION OF THE INVENTION

The present invention is predicated, in part, on the determination that the incidence of unwanted amplicon generation, deriving from the binding of primers to DNA regions other than the region of interest, can be minimised by tagging and rendering functionally inefficient the primer which is likely to result in primer hybridisation and extension at a site which is not the site of interest. The method of the present invention therefore provides a simple and efficient means of amplifying a nucleic acid region of interest.

Accordingly, one aspect of the present invention provides a method of amplifying a nucleic acid region of interest, said method comprising:
(i) contacting a nucleic acid sample with:
   (a) one or more forward primers directed to said region of interest; and
   (b) one or more reverse primers directed to said region of interest
   wherein the primers of either group (a) or group (b) are functionally inefficient and are operably linked at their 5′ end to an oligonucleotide tag;
(ii) amplifying the nucleic acid sample of step (i) through at least two cycles of amplification;
(iii) contacting the amplified nucleic acid of step (ii) with a primer which is directed to part or all of the sequence which is complementary to that of the oligonucleotide tag of step (i); and
(iv) amplifying the nucleic acid sample of step (iii).

Reference to a nucleic acid “region of interest” should be understood as a reference to any region of DNA or RNA which is sought to be amplified. This may be a gene or part of a gene. To this end, reference to “gene” should be understood as a reference to a DNA molecule which codes for a protein product, whether that be a full length protein or a protein fragment. In terms of chromosomal DNA, the gene will include both intron and exon regions. However, to the extent that the DNA of interest is cDNA, such as might occur if the DNA of interest is vector DNA or reverse transcribed mRNA, there may not exist intron regions. Such DNA may nevertheless include 5′ or 3′ untranslated regions. Accordingly, reference to “gene” herein should be understood to encompass any form of DNA which codes for a protein or protein fragment including, for example, genomic DNA and cDNA. The subject nucleic acid region of interest may also be a non-coding portion of genomic DNA which is not known to be associated with any specific gene (such as the commonly termed “junk” DNA regions). It may be any region of genomic DNA produced by recombination, either between 2 regions of genomic DNA or 1 region of genomic DNA and a region of foreign DNA such as a virus or an introduced sequence. It may be a region of a partly or wholly synthetically or recombinantly generated nucleic acid molecule. The subject nucleic acid sequence of interest may also be a region of DNA which has been previously amplified by any nucleic acid amplification method, including PCR (i.e. it has been generated by an amplification method).

The subject “nucleic acid” region may be DNA or RNA or derivative or analogue thereof. Where the region of interest is a DNA sequence which encodes a proteinaceous molecule it may take the form of genomic DNA, cDNA which has been generated from a mRNA transcript, or DNA generated by nucleic acid amplification. However where the subject DNA does not encode a protein, either genomic DNA or synthetically or recombinantly generated DNA may be the subject of analysis. As would be appreciated by the skilled person, both synthetically and recombinantly generated DNA may also encode all or part of a protein. However, if the subject method is directed to detecting a region of RNA, it would be appreciated that it will first be necessary to reverse transcribe the RNA to DNA, such as using RT-PCR. The subject RNA may be any form of RNA, such as mRNA, primary RNA transcript, ribosomal RNA, transfer RNA, micro RNA or the like. Preferably, said nucleic acid region of interest is a DNA region of interest. To this end, said DNA includes DNA generated by reverse transcription from RNA which is ultimately the subject of analysis, and DNA generated by a nucleic acid amplification method such as PCR.

The present invention therefore more preferably provides a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) are functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

Reference to “DNA” should be understood as a reference to deoxyribonucleic acid or derivative or analogue thereof. In this regard, it should be understood to encompass all forms of DNA, including cDNA and genomic DNA. The nucleic acid molecules of the present invention may be of any origin including naturally occurring (such as would be derived from a biological sample), recombinantly produced or synthetically produced.

Reference to “derivatives” should be understood to include reference to fragments, homologs or orthologs of said DNA from natural, synthetic or recombinant sources. “Functional derivatives” should be understood as derivatives which exhibit any one or more of the functional activities of DNA. The derivatives of said DNA sequences include fragments having particular regions of the DNA molecule fused to other proteinaceous or non-proteinaceous molecules. “Analogs” contemplated herein include, but are not limited to, modifications to the nucleotide or nucleic acid molecule such as modifications to its chemical makeup or overall conformation. This includes, for example, incorporation of novel or modified purine or pyrimidine bases or modification to the manner in which nucleotides or nucleic acid molecules interact with other nucleotides or nucleic acid molecules such as at the level of backbone formation or complementary base pair hybridisation. The biotinylation or other form of labelling of a nucleotide or nucleic acid molecules is an example of a “functional derivative” as herein defined.

Preferably, said DNA is a gene or gene fragment, a chromosomal gene translocation breakpoint or DNA produced by prior nucleic acid amplification.

According to this aspect, the present invention provides a method of amplifying a gene or gene fragment of interest, said method comprising:
(i) contacting a DNA sample with:
   (a) one or more forward primers directed to said gene or gene fragment of interest; and
   (b) one or more reverse primers directed to said gene or gene fragment of interest
   wherein the primers of either group (a) or group (b) are functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

In another aspect, there is provided a method of amplifying a chromosomal gene translocation breakpoint region of interest, said method comprising:
(i) contacting a DNA sample with:
   (a) one or more forward primers directed to said chromosomal gene translocation breakpoint region of interest; and
   (b) one or more reverse primers directed to said chromosomal gene translocation breakpoint region of interest
   wherein the primers of either group (a) or group (b) are functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

In yet another aspect, there is provided a method of amplifying DNA produced by prior nucleic acid amplification, said method comprising:
(i) contacting a DNA sample with:
   (a) one or more forward primers directed to said chromosomal gene translocation breakpoint region of interest; and
   (b) one or more reverse primers directed to said chromosomal gene translocation breakpoint region of interest
   wherein the primers of either group (a) or group (b) are functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

Preferably, said prior nucleic acid amplification is PCR.

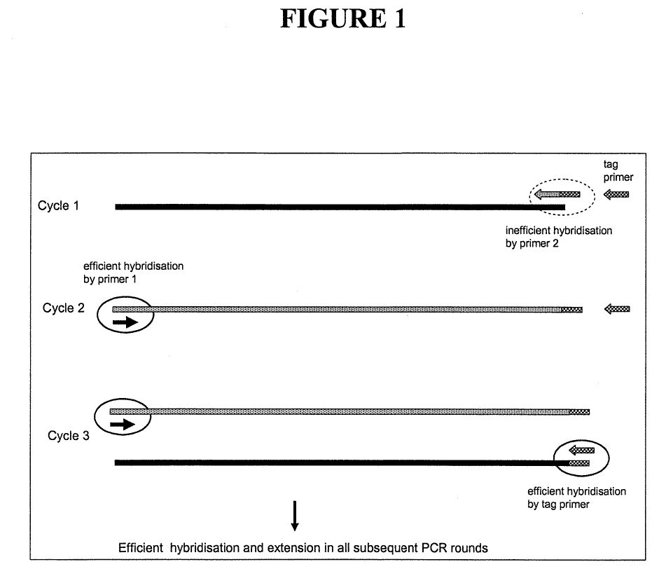

In a classical PCR, the primers and reaction conditions are designed so that primer hybridisation and extension of the forward and reverse primers occur at or close to the maximum efficiency so that the number of amplicons approximately doubles with each cycle resulting in efficient exponential amplification. The method of the present invention, however, is predicated on the use of forward and reverse primer sets where the primers of one set have been designed or are otherwise used under conditions wherein they do not hybridise and extend efficiently. Accordingly, although the efficient primer set will amplify normally, the inefficient set will not. As a consequence, when a sequence of interest is amplified, the number of amplicon strands is significantly less than that which would occur in a classical PCR. Efficient amplification only commences once amplicons have been generated which incorporate, at one end, the tag region of the inefficient primer (generation of these amplicons is illustrated in FIG. 1 and the functioning of the tag is discussed in more detail hereinafter). At this point, the primers directed to the tag regions effect a normal amplification rate. A “bottleneck” is therefore effectively created in terms of the generation of transcripts from the inefficient primer set.

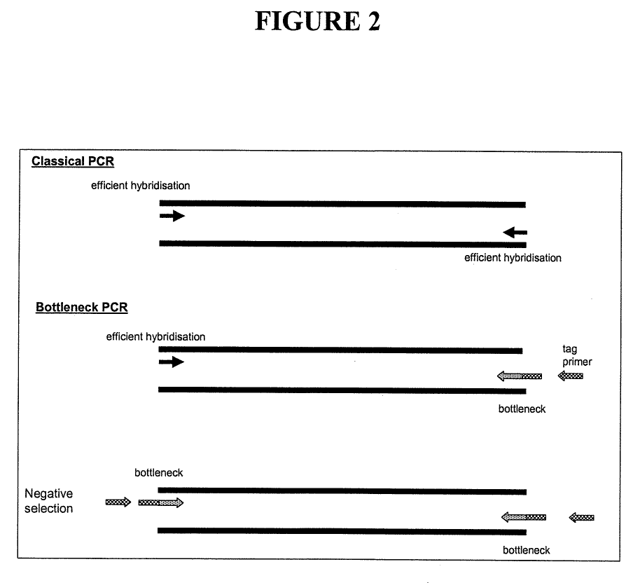

A more severe bottleneck is usefully created where the primers which are rendered inefficient are degenerate and hybridise widely, or are directed to commonly repeated sequences, such as an alu sequence. Amplification of unwanted product may result if the primer binding sites are closely apposed and if the inefficient primers can act as forward primers and reverse primers. However, owing to both primers being inefficient, amplification is initially extremely inefficient and there is a severe bottleneck. This situation is illustrated in FIG. 2. Efficient amplification only commences once amplicon strands have been generated which comprise the tag region of the inefficient primer at one end and its complement at the other. After any given number of cycles, the number of such amplicons is, however, substantially less than that which occurs during amplification of the sequence of interest, as described above. The amount of unwanted product at the end of the amplification reaction is correspondingly reduced.

Hybridisation and extension of an inefficient primer which has correctly hybridised to the sequence of interest followed in a subsequent cycle by hybridisation and extension of an efficient primer to the previously synthesised amplicon generates a template to which the tag primer can efficiently hybridise and extend. Since such molecules together with their complements provide upstream and downstream binding sites, each for an efficient primer (the tag primer and one member of the efficient set), succeeding cycles of amplification from such templates are both efficient and exponential. The result is that, after an initial lag or “bottleneck”, the overall rate of amplification speeds up in later cycles so that a near doubling of amplicon number with each cycle results. However, the net result is that there is negative selection against amplification of undesired amplicons as compared to amplicons of the sequence of interest, owing to the bottleneck at each end for the former and only at one end for the latter.

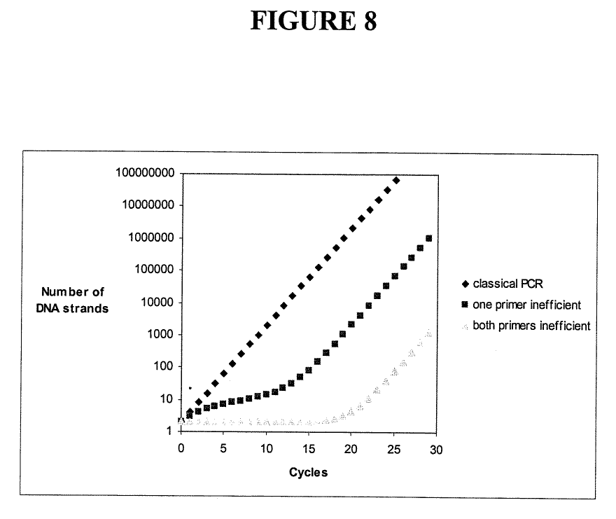

Accordingly, if the same number of commencing target sequences is considered and comparison to the amplification produced by classical PCR is made, application of the method of the present invention will produce a lesser increase in the number of amplicons of the sequence of interest and an even lesser increase in the number of amplicons of unwanted sequences, as illustrated in FIG. 8. Although amplification of both wanted and unwanted products occurs, there is relative enrichment of the sequence of interest relative to the unwanted sequences. There is an inverse relationship between absolute amplification and enrichment since decreasing the efficiency of the inefficient primer set produces increased enrichment at the expense of lesser amplification.

Those skilled in the art will appreciate that the amplifications of the first and second phases of the method do not need to be performed as physically separate reactions but can be simply and conveniently performed in the same reaction container; the first phase commences with cycle 1 and proceeds thereafter whereas the second phase commences at cycle 3 and proceeds thereafter.

In terms of deciding whether it should be the forward primer set or the reverse primer set which is rendered inefficient, this may vary from one situation to the next. In general, however, it is likely that one would seek to render inefficient the hybridisation and extension of the primer set which exhibits the greatest probability of binding to sequences other than the target of interest. In terms of the exemplification provided herein, the experimental design for the enrichment of the BCR-ABL breakpoint is based on rendering inefficient the ABL primer set rather than the BCR primer set. In yet another example, if the primer target sequence of a DNA region of interest is known on only one side of the DNA region, a universal or degenerate primer could be used to initiate primer extension and amplification from the other side. However, since such a primer would, by definition, bind promiscuously across the sample DNA, by rendering this primer set inefficient, an enhanced amplification of the DNA region of interest compared to that of irrelevant sequences can be achieved. In still another example, in terms of deciding whether it should be the forward primer set or the reverse primer set which is rendered inefficient when the DNA sample being amplified has been produced by prior nucleic acid amplification, one might seek to render inefficient the hybridisation and extension of the forward primer(s) if the forward primer(s) in the initial commencing amplification were promiscuous or, conversely, the hybridisation and extension of the reverse primer(s) if the reverse primer(s) in the initial commencing amplification were promiscuous

Preferably, the primer group which is rendered inefficient is the group which has the potential to hybridise promiscuously to non-target DNA regions.

According to this aspect, there is provided a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) are functionally inefficient and are operably linked at their 5′ end to an oligonucleotide tag and wherein said functionally inefficient primer group is the primer group which has the potential to hybridise promiscuously to non-target DNA regions;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

Reference to a “primer” or an “oligonucleotide primer” should be understood as a reference to any molecule comprising a sequence of nucleotides, or functional derivatives or analogues thereof, the function of which includes hybridisation to a region of a nucleic acid molecule of interest (the DNA of interest interchangeably being referred to as a “target DNA”). It should be understood that the primer may comprise non-nucleic acid components. For example, the primer may also comprise a non-nucleic acid tag such as a fluorescent or enzymatic tag or some other non-nucleic acid component which facilitates the use of the molecule as a probe or which otherwise facilitates its detection or immobilisation. The primer may also comprise additional nucleic acid components, such as the oligonucleotide tag which is discussed in more detail hereinafter. In another example, the primer may be a protein nucleic acid which comprises a peptide backbone exhibiting nucleic acid side chains. Preferably, said oligonucleotide primer is a DNA primer.

Reference to “forward primer” should be understood as a reference to a primer which amplifies the target DNA in the DNA sample of interest by hybridising to the antisense strand of the target DNA.

Reference to “reverse primer” should be understood as a reference to a primer which amplifies the target DNA in the DNA sample of interest and in the PCR by hybridising to the sense strand of the target DNA.

The design and synthesis of primers suitable for use in the present invention would be well known to those of skill in the art. In one embodiment, the subject primer is 4 to 60 nucleotides in length, in another embodiment 10 to 50 in length, in yet another embodiment 15 to 45 in length, in still another embodiment 20 to 40 in length, in yet another embodiment 25 to 35 in length. In yet still another embodiment, primer is about 26, 27, 28, 29, 30, 31, 32, 33 or 34 nucleotides in length.

In terms of the number of primers which are used in the method of the invention, this can be determined by the person of skill in the art. If the sequences of the two ends of the sequence of interest are known then only one forward and one reverse primer may be needed, but if this information is not available then multiple forward and/or reverse primers or one or more degenerate primers may be employed. For isolation of translocation breakpoints, which may occur at unknown points within large regions of the interacting genes, multiple primers may be used in an attempt to ensure that at least 1 forward and 1 reverse primer closely span the breakpoint so that efficient PCR amplification will occur. With regard to chronic myeloid leukaemia (CML), for example, nearly all BCR translocations involve one of two regions, each of approximately 3 kb in length. In this case, 12 outer forward primers may be used. The ABL gene, however, is larger, the region commonly involved in translocation is approximately 140 kb in length, and up to 282 or more outer reverse primers may be used. In one particular embodiment, a combination of 6 forward primers and 24 reverse primers is used and in another embodiment a combination of 1 forward primer and 282 reverse primers. In yet another combination there is 6 forward primers and 270-310 reverse primers. In yet another combination there is 1-20 forward primers and 250-350 reverse primers. The primer number which is selected to be used will depend on the target region of interest and thus may vary from one target to the next. As would be understood by the person of skill in the art, in terms of classical PCR a large number of primers in each individual PCR reaction increases the probability of non-specific amplification reactions. The method of the present invention, however, enables the use of a larger number of primers due to the minimisation of non-specific amplification reactions by virtue of rendering one primer set functionally inefficient.

It should also be understood that to the extent that there are 2 or more different primers within a single forward or reverse primer pool, one can render all the primers within that pool inefficient or one can render inefficient only a select subgroup of these primers which are thought to be the most problematic in terms of generating irrelevant amplicons.

By “functionally inefficient” is meant that the subject primer has been modified and/or is utilised under environmental conditions which render its hybridisation and extension less effective in terms of the number and/or rate of amplicon generation from that primer than if the design of the primer had not been modified or it had not been used under the subject environmental conditions. Methods of rendering a primer functionally inefficient would be well known to the person of skill in the art and include but are not limited to:
(i) reducing the concentration of primer which is used;
(ii) reducing the hybridisation time of the primer;
(iii) increasing the temperature at which the hybridisation reaction is required to occur;
(iv) modifying the length of the primer;
(v) altering the primer melting temperature;
(vi) introducing a chaotropic agent during the hybridisation/primer extension phase;
(vii) introducing into the reaction a competitive inhibitor to the primer, such as its complementary sequence;
(viii) introducing nucleotide mismatches into the primer sequence;
(ix) using primer analogs which hydrogen-bond less efficiently in the context of hybridisation.

It would be appreciated that in addition to potentially modifying the primer itself, one can alternatively (or additionally) elect to modify the reaction conditions to achieve the same outcome. To this end, it should be appreciated that one could also design a system which uses two or more of the above-listed methods to achieve the functional inefficiency of one primer without similarly rendering inefficient the other primer. This is more likely to become an issue where one elects to modify the reaction conditions rather than the primer itself. For example, if one elects to increase reaction temperature in order to reduce efficiency, this will affect the functionality of both the primer groups (i.e. the forward primers and the reverse primers). Accordingly, to minimise how far the temperature is required to be increased, one may combine this with the use of a primer which has been increased in length in order to maximise the inefficiency of one primer but not the other. In yet another example, one may choose not to alter reaction conditions but, instead, may reduce the concentration of primer which is utilised. In another example, one may reduce the length of the primer or reduce its concentration in combination with a reduced hybridisation time. Designing and altering these factors to achieve functional inefficiency would be well known to the person of skill in the art since they are issues which are routinely considered and well described in the art in the context of designing PCR reaction (Sambrook J. and Russell D. “Molecular cloning: a laboratory manual” 3rd edition published by Cold Spring Harbor, 2001), albeit usually in the context of reducing the possibility of the functional inefficiency of the forward and reverse primers as opposed to deliberately inducing this state. Nevertheless, the considerations which are required to be made in order to design an efficient PCR reaction are the same as the ones which are made to design part of the reaction to function inefficiently. Accordingly, the issue is merely one of how these considerations will be applied. To this end, it should also be appreciated that the notion of rendering a primer functionally inefficient encompasses not just modifying the design of the primer itself but also or alternatively modifying the reaction conditions within which the primer is required to function.

Preferably, the functional inefficiency is hybridisation inefficiency which is achieved by one or more of modifying primer length, sequence, annealing temperature, starting concentration or hybridisation time.

According to this aspect, there is provided a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) hybridise inefficiently and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i); and
(iv) amplifying the DNA sample of step (iii).

Preferably, said functionally inefficient primer group is the primer group which, in the absence of having been rendered functionally inefficient has the potential to hybridise promiscuously to non-target DNA regions.

In another preferred embodiment, said DNA region of interest is a gene or gene fragment or a chromosomal gene translocation breakpoint.

As detailed hereinbefore, the method of the present invention is aimed at enriching a sequence of interest and is predicated on rendering either the forward or reverse primer group inefficient. As a consequence, amplification of the sequence of interest is inefficient, but it is much more inefficient for other unwanted sequences which are amplified by one or more of the members of the inefficient primer group acting as both forward and reverse primers, This situation, which is exemplified in FIG. 2, results in negative selection against such unwanted sequences and enrichment of the sequence of interest. However, it is also desirable to revert to efficient levels of amplification in order to facilitate increasing the copy number of the amplicon of interest. This is conveniently achieved by incorporating into the functionally inefficient primer a tag which can itself function to generate a primer hybridisation target site. By this means there is effectively enabled the ability to effect efficient amplification from the same direction as the functionally inefficient primer.

The generation of such templates, which enable efficient priming by the tag primer in later cycles, results in further enrichment, and the sequence of events is shown in FIG. 1.

Primer 2, which comprises an oligonucleotide tag, hybridises to a template strand, which is either an original genomic strand or an amplicon strand generated in a previous PCR cycle. This primer hybridises inefficiently but it does nevertheless generate some complementary strands during this first cycle. In a second cycle of amplification, primer 1 hybridises to the primer 2 generated amplicon strands and extends, generating a strand comprising a binding site for the tag primer, which is able to be efficiently amplified by the tag primer in later cycles. The Figure illustrates the situation where primer 1 is an efficient primer which selects for those amplicons which correspond to the DNA region of interest and which efficiently generates amplicon strands containing a binding site for the tag primer. For situations characterised by forward and reverse priming by one or more members of the group of inefficient primers, such as amplification of common sequences such as Alu or sequences able to be amplified by a degenerate or universal primer, and for which amplification is undesired, the primer corresponding to primer 1 in FIG. 1 is inefficient and its extension in the second cycle generates fewer strands containing the binding site for the tag primer. Accordingly, there effectively occurs a further negative selection against production of such undesired templates and a positive selection in favour of template strands of the sequence of interest, which comprise primer 1 at one end, and the binding site for the tag at the other end.

In order to ensure that these oligonucleotide tags do not interfere with the extension of the primer, the primers are linked to the oligonucleotide tag at their 5′ end. Reference to “oligonucleotide tag” should therefore be understood as a reference to a nucleotide sequence of usually less than 50 nucleotides which is linked to the 5′ end of the functionally inefficient primer of the present invention. In one embodiment, the tag is 25-30 bases in length. It should also be understood that consistently with the definitions provided in relation to the forward and reverse primers, the oligonucleotide tags herein described may also comprise non-nucleic acid components such as isolation or visualisation tags eg. biotin, enzymatic labels, fluorescent labels and the like. This enables quick and simple isolation or visualisation of the tagged primers or amplicons via non-molecular methods.

That the oligonucleotide tag is “operably linked” to the primer should be understood as a reference to those regions being linked such that the functional objectives of the tagged primer, as detailed hereinbefore, can be achieved. In terms of the means by which these regions are linked and, further, the means by which the subject oligonucleotide primer binds to its target DNA region, these correspond to various types of interactions. In this regard, reference to “interaction” should be understood as a reference to any form of interaction such as hybridisation between complementary nucleotide base pairs or some other form of interaction such as the formation of bonds between any nucleic or non-nucleic acid portion of the primer molecule or tag molecule with any other nucleic acid or non-nucleic acid molecule, such as the target molecule, a visualisation means, an isolation means or the like. This type of interaction may occur via the formation of bonds such as, but not limited to, covalent bonds, hydrogen bonds, van der Wals forces or any other mechanism of interaction. Preferably, to the extent that the interaction occurs between the primer and a region of the target DNA, said interaction is hybridisation between complementary nucleotide base pairs. In order to facilitate this interaction, it is preferable that the target DNA is rendered partially or fully single stranded for a time and under conditions sufficient for hybridisation with the primer to occur.

It should be understood that the oligonucleotide primers and tags of the present invention should not be limited to the specific structure exemplified herein (being a linear, single-stranded molecule) but may extend to any suitable structural configuration which achieves the functional objectives detailed herein. For example, it may be desirable that all or part of the oligonucleotide is double stranded, comprises a looped region (such as a hairpin bend) or takes the form of an open circle confirmation, that is, where the nucleotide primer is substantially circular in shape but its terminal regions do not connect.

Facilitating the interaction of the nucleic acid primer with the target DNA may be performed by any suitable method. Those methods will be known to those skilled in the art. To this end, it should be understood that the primer directed to the tag can be incorporated into the reaction tube at any suitable time point. For example, it may be incorporated prior to the commencement of the initial amplification cycles, that is together with the forward and reverse primer sets, or it may be incorporated subsequently to the initial two amplification cycles. In either case, the primer to the tag region will become functional only after amplicons have been generated which incorporate the tag region, as hereinbefore described.

Methods for achieving primer directed amplification are also very well known to those of skill in the art. In a preferred method, said amplification is polymerase chain reaction, NASBA or strand displacement amplification. Most preferably, said amplification is polymerase chain reaction.

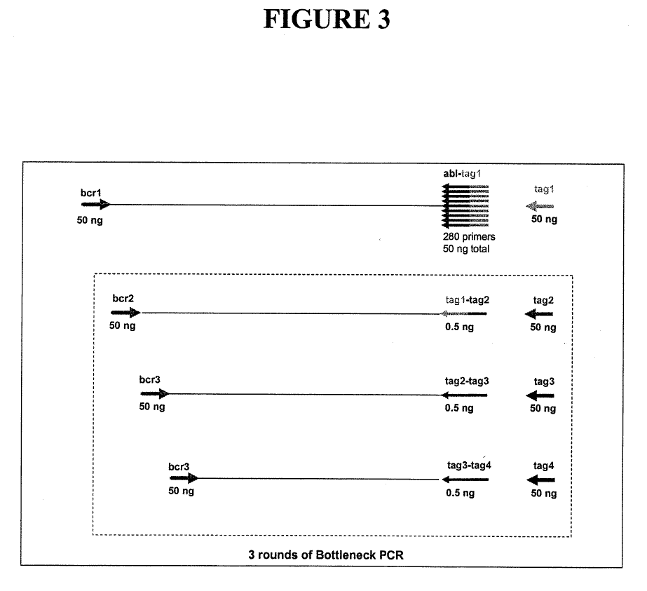

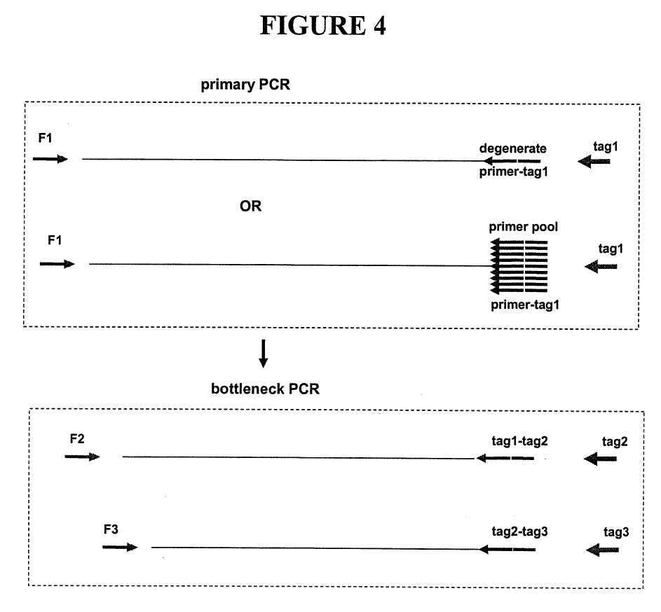

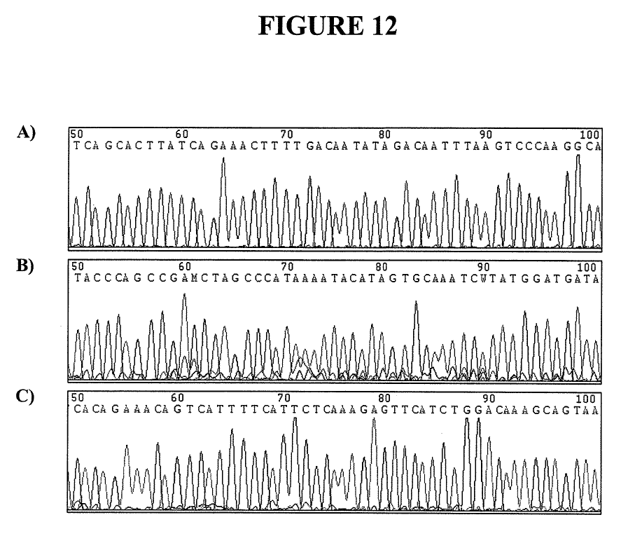

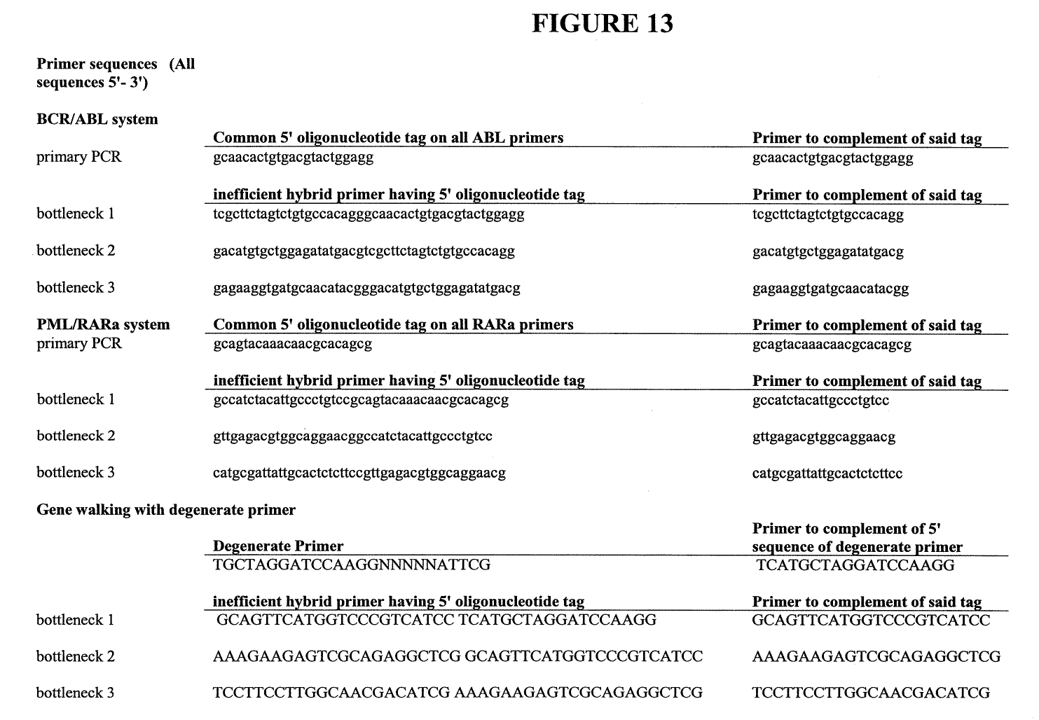

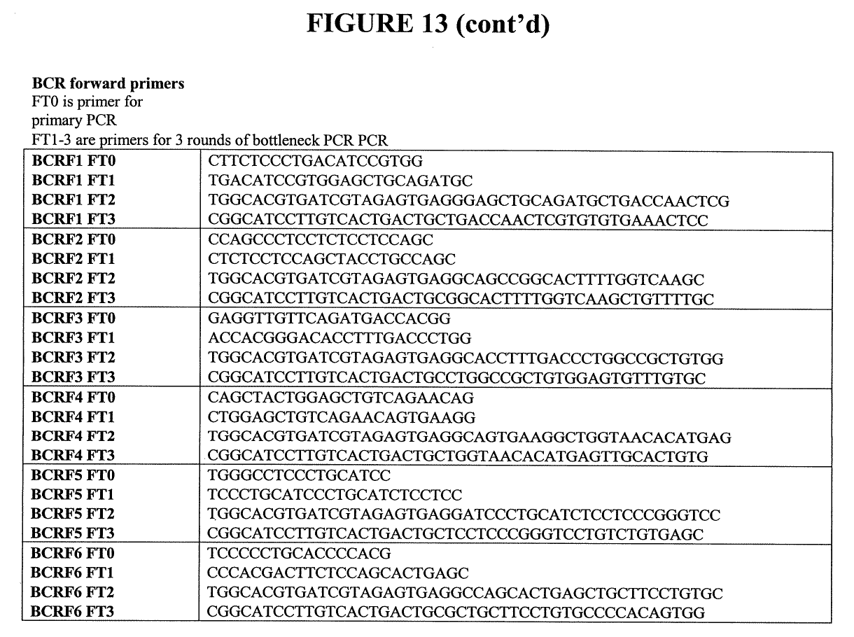

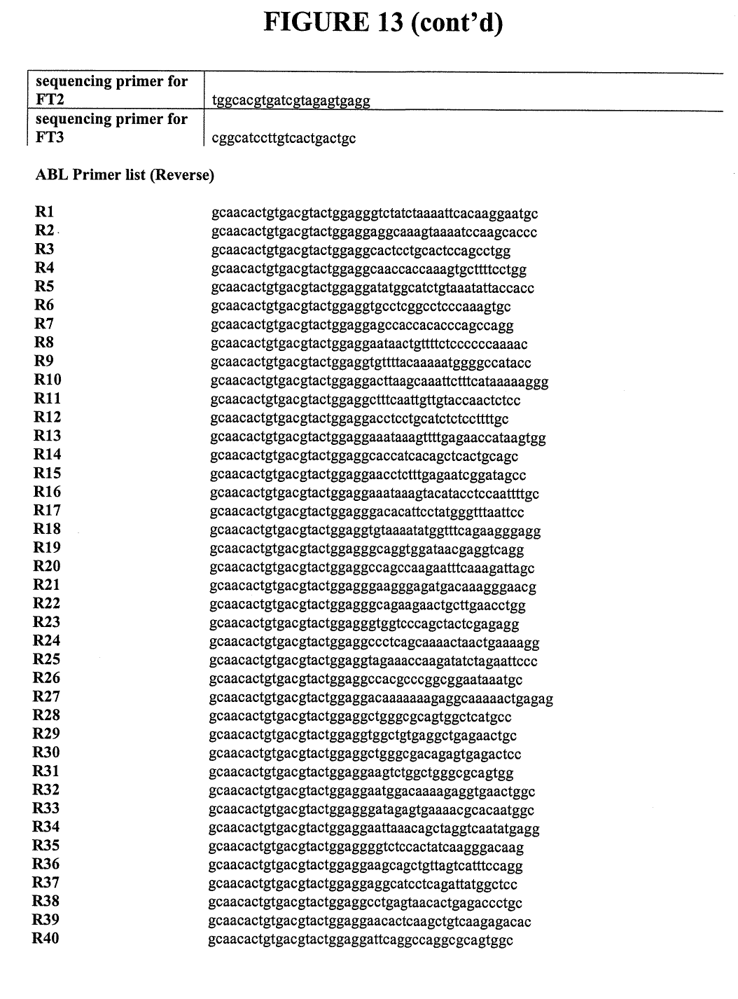

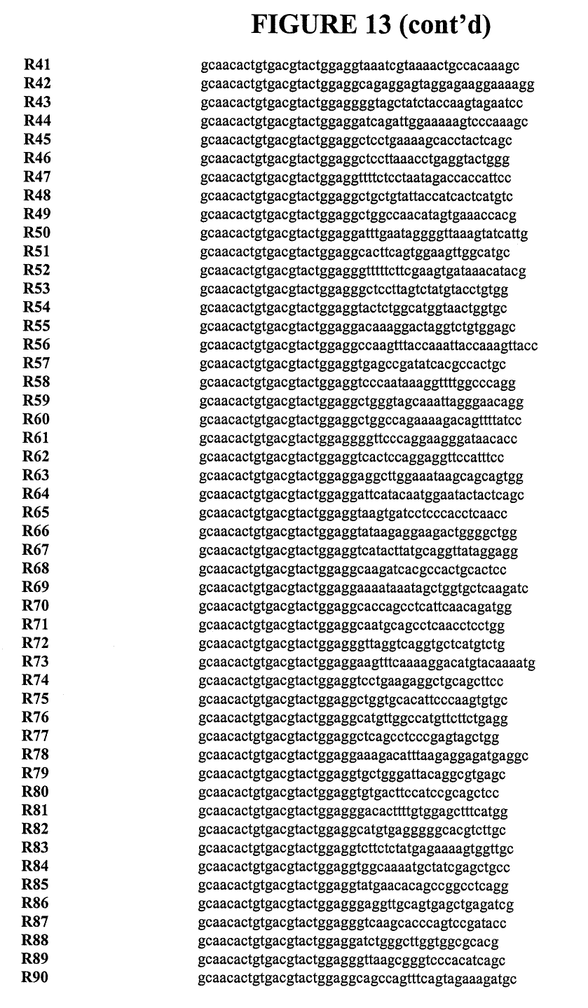

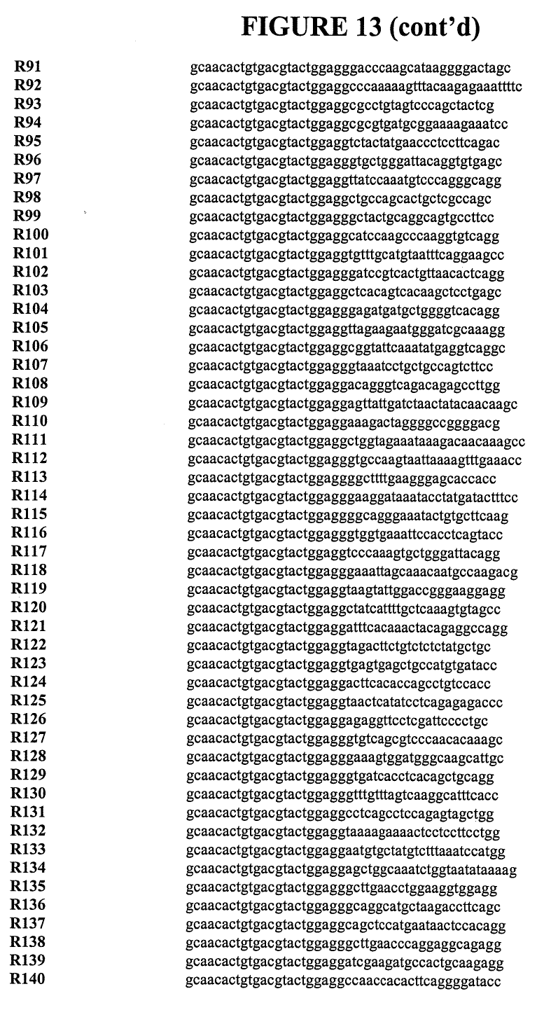

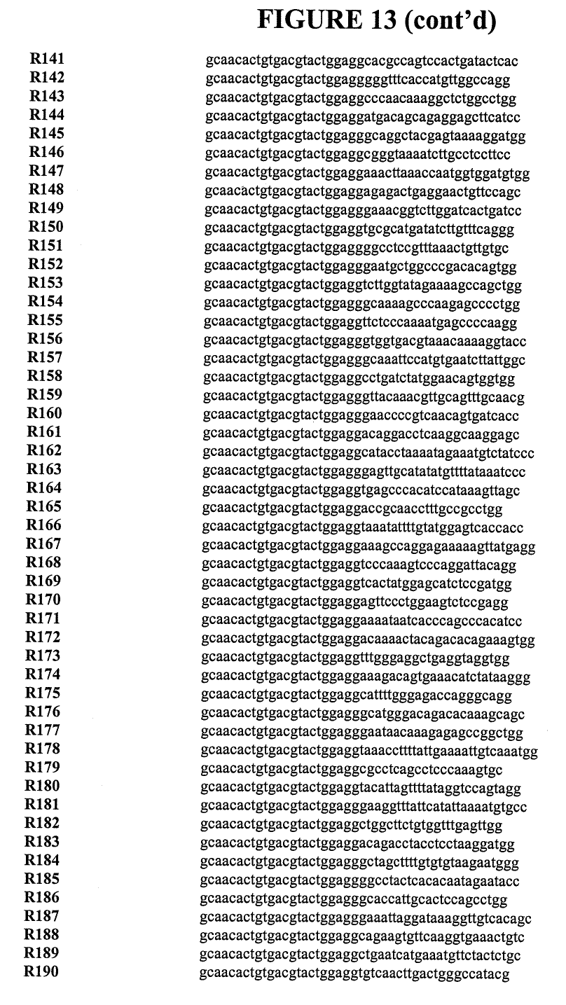

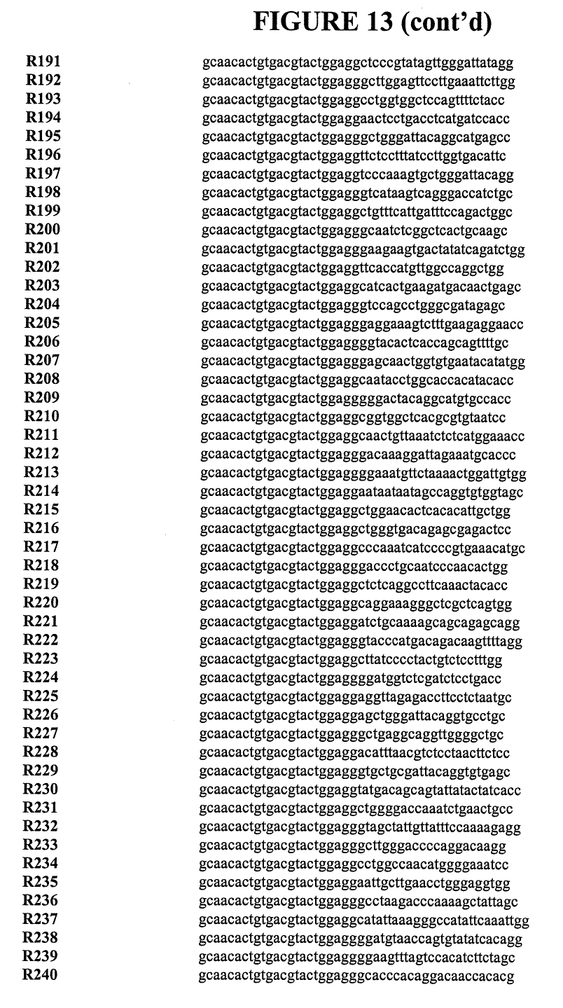

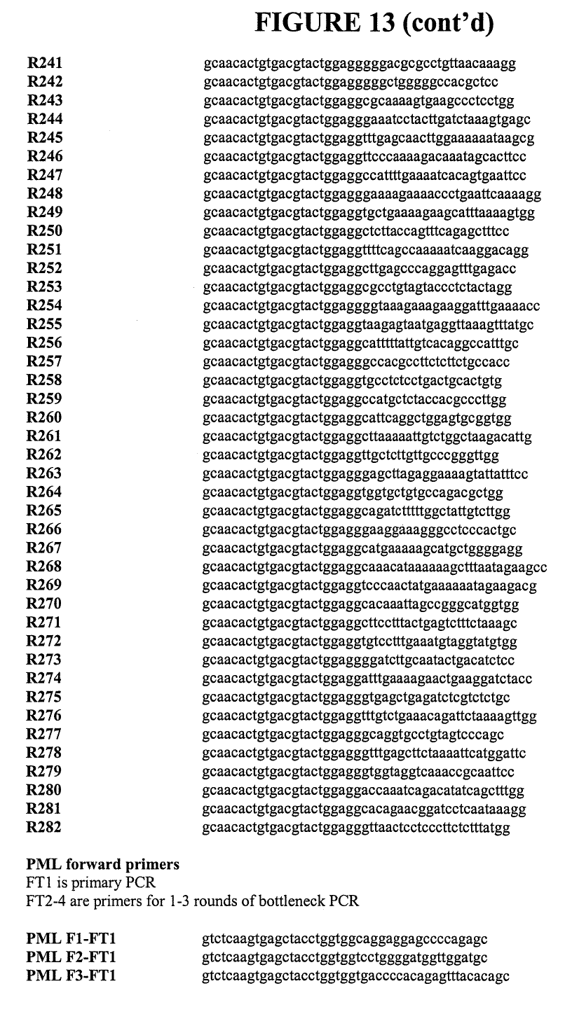

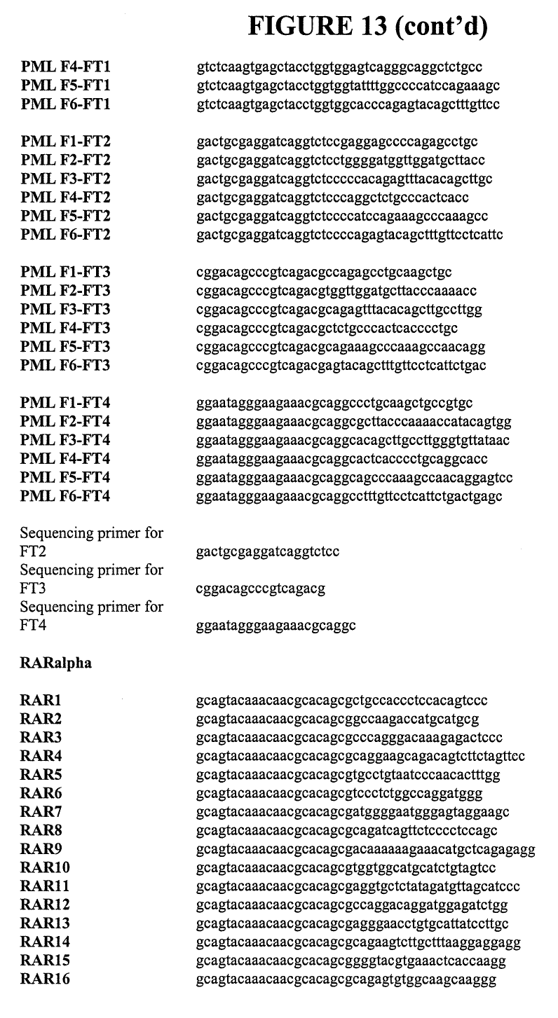

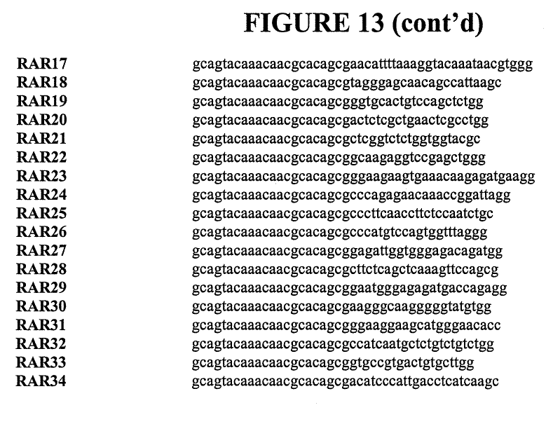

Methods for performing serial nucleic acid amplification, utilising product from a prior amplification as template for a subsequent reaction, are also very well known to those of skill in the art. In another preferred method, as performed for example to produce the results shown in FIGS. 3-12, a bottleneck amplifications may be performed to produce selective amplification of a DNA sequence of interest which has been produced by either a prior bottleneck PCR or a prior non-selective PCR. Those with skill in the art will appreciate that sequential bottleneck PCRs can be performed.

There is therefore provided a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) hybridise inefficiently and are operably linked at their 5′ end to an oligonucleotide tag;
(ii) amplifying the DNA sample of step (i) through at least two cycles of PCR;
(iii) contacting the amplified DNA of step (ii) with a primer which is directed to part or all of the sequence which is complementary to that of the oligonucleotide tag of step (i); and
(iv) amplifying the DNA sample of step (iii) by PCR.

Preferably, said DNA region of interest has been produced by prior nucleic acid amplification.

In another preferred embodiment, said DNA region of interest is a gene or gene fragment or a chromosomal gene translocation breakpoint such as the BCR ABL breakpoint.

In terms of effecting the method of the present invention, it should be appreciated that the primers of steps (i) and (iii) can be simultaneously added to the reaction solution prior to the first two amplification cycles, or the primers of step (iii) can be introduced after the first or second cycle of amplification. This will depend on how the skilled person is seeking to perform the PCR reaction. For example, for ease of use, it is often desirable to be able to perform the entire reaction in a single tube. Nevertheless, any other method of achieving the steps of the invention can be used.

Reference to a “sample” should be understood as a reference to either a biological or a non-biological sample. Examples of non-biological samples includes, for example, the nucleic acid products of synthetically produced nucleic acid populations. Reference to a “biological sample” should be understood as a reference to any sample of biological material derived from an animal, plant or microorganism (including cultures of microorganisms) such as, but not limited to, cellular material, blood, mucus, faeces, urine, tissue biopsy specimens, fluid which has been introduced into the body of an animal and subsequently removed (such as, for example, the saline solution extracted from the lung following lung lavage or the solution retrieved from an enema wash), plant material or plant propagation material such as seeds or flowers or a microorganism colony. The biological sample which is tested according to the method of the present invention may be tested directly or may require some form of treatment prior to testing. For example, a biopsy sample may require homogenisation prior to testing or it may require sectioning for in situ testing. Further, to the extent that the biological sample is not in liquid form, (if such form is required for testing) it may require the addition of a reagent, such as a buffer, to mobilise the sample.

To the extent that the target DNA is present in a biological sample, the biological sample may be directly tested or else all or some of the nucleic acid material present in the biological sample may be isolated prior to testing. It is within the scope of the present invention for the target nucleic acid molecule to be pre-treated prior to testing, for example inactivation of live virus or being run on a gel. It should also be understood that the biological sample may be freshly harvested or it may have been stored (for example by freezing) prior to testing or otherwise treated prior to testing (such as by undergoing culturing).

Reference to “contacting” the sample with the primer should be understood as a reference to facilitating the mixing of the primer with the sample such that interaction (for example, hybridisation) can occur. Means of achieving this objective would be well known to those of skill in the art.

The choice of what type of sample is most suitable for testing in accordance with the method disclosed herein will be dependent on the nature of the situation, such as the nature of the condition being monitored. For example, in a preferred embodiment a neoplastic condition is the subject of analysis. If the neoplastic condition is a leukaemia, a blood sample, lymph fluid sample or bone marrow aspirate would likely provide a suitable testing sample. Where the neoplastic condition is a lymphoma, a lymph node biopsy or a blood or marrow sample would likely provide a suitable source of tissue for testing. Consideration would also be required as to whether one is monitoring the original source of the neoplastic cells or whether the presence of metastases or other forms of spreading of the neoplasia from the point of origin is to be monitored. In this regard, it may be desirable to harvest and test a number of different samples from any one mammal.

Choosing an appropriate sample for any given detection scenario would fall within the skills of the person of ordinary skill in the art.

The term “mammal” to the extent that it is used herein includes humans, primates, livestock animals (e.g. horses, cattle, sheep, pigs, donkeys), laboratory test animals (e.g. mice, rats, rabbits, guinea pigs), companion animals (eg. dogs, cats) and captive wild animals (eg. kangaroos, deer, foxes). Preferably, the mammal is a human or a laboratory test animal. Even more preferably the mammal is a human.

As detailed hereinbefore, the method of the present invention is preferably performed as a sequential series of amplification cycles. To this end, a minimum of two cycles of amplification is required at step (ii) in order to effect the generation of amplicons which are suitable to undergo amplification by the primer to the oligonucleotide tag of the inefficient primer and the original primer which retained efficiency. It should be understood, however, that the method of the invention may be designed to conduct 3 or more amplification cycles before contact with the primer to the oligonucleotide tag is effected. Alternatively, if all the reagents of the present method are introduced to the reaction tube prior to commencement, more than two cycles of amplification before the tag-based amplification of step (iv) becomes effective may not occur. It should also be understood that, even within the same PCR, each amplification cycle may generate new amplicons which are then amenable to subsequent tag-based amplification.

Although the method of the present invention has been designed such that the rounds of amplification can be sequentially performed directly on the amplification product of a previous round of amplification, this should not be understood as a limitation in terms of whether any additional steps are sought to be incorporated by the skilled person, such as enrichment/selection steps. For example, one may seek to select or enrich for the desired amplicons after the first round of amplification and to thereafter conduct the second round of amplification on that material alone. Methods which one could utilise to select or enrich include:
(i) a selection step based on the unique markers which are linked to the primers. For example, biotinylation of one of the primers provides means of identifying and isolating amplicons which have resulted from extension by either the forward or reverse primers. By flooding the amplification product with biotinylated primer, the primer can act as a probe to identify the amplicons of interest and the biotinylation can provide a basis for isolating those amplicons. By ensuring that each of the primer groups of the present invention comprises a unique tag, it is possible to select out, with significant particularity, only specific amplicons of interest. In particular, the skilled person would seek to exclude amplicons which have been amplified by a forward primer but which have not then been amplified by a reverse primer.
(ii) running the products on a gel and excising out only certain bands or regions which are likely to be relevant and thereafter subjecting these to a further amplification step. When a band is present on the gel after the second cycle amplification, if there are any problems in sequencing an attempt can be made to clean it up by cutting the product out of the gel and performing a series of PCR reactions using individual primers and/or smaller pools of primers.

Although the method of the present invention may be adapted to include any such additional steps, one of the unique advantages of the present method is that it has been designed in order to minimise the generation of irrelevant amplicons, thereby minimising the need to implement enrichment or selection steps. Nevertheless, depending on the particular situation, the incorporation of such steps may nevertheless be useful.

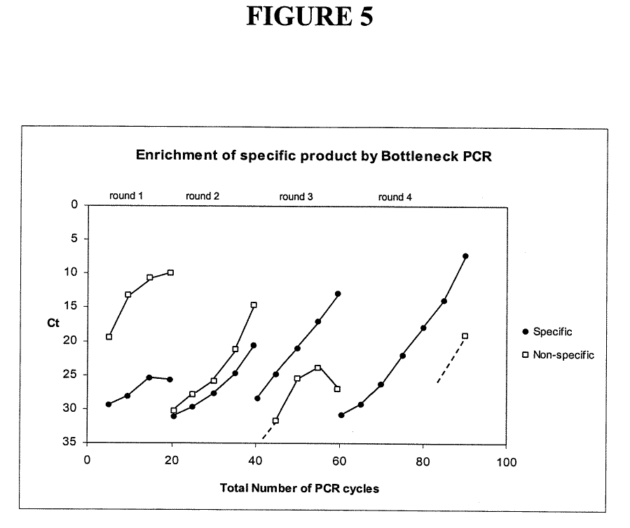

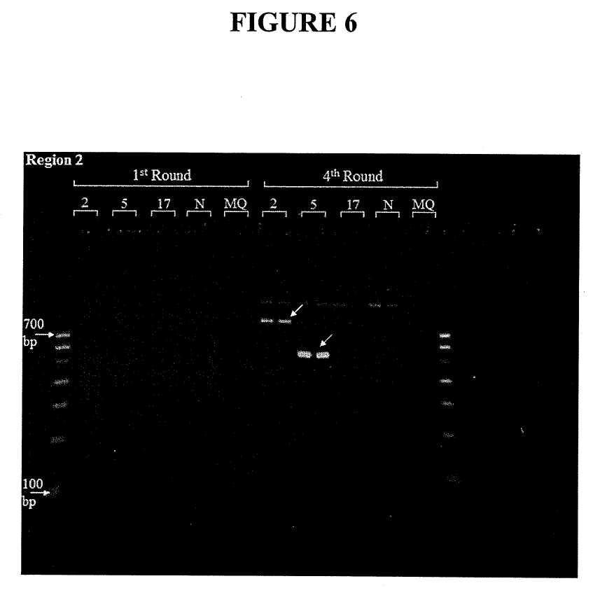

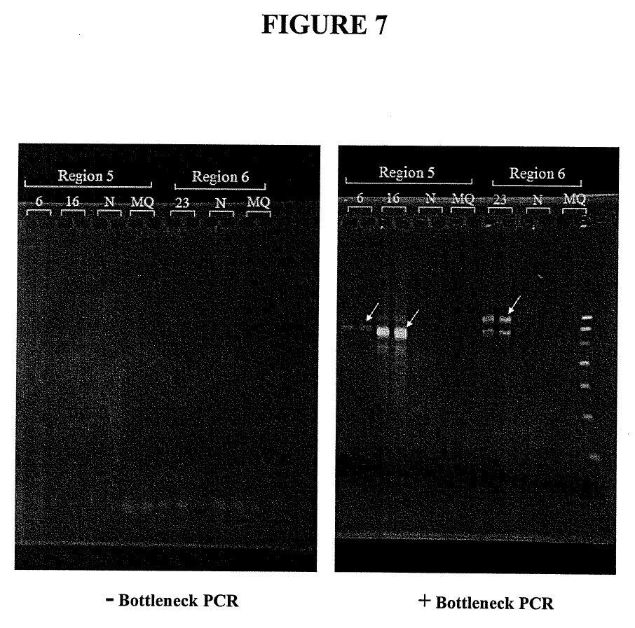

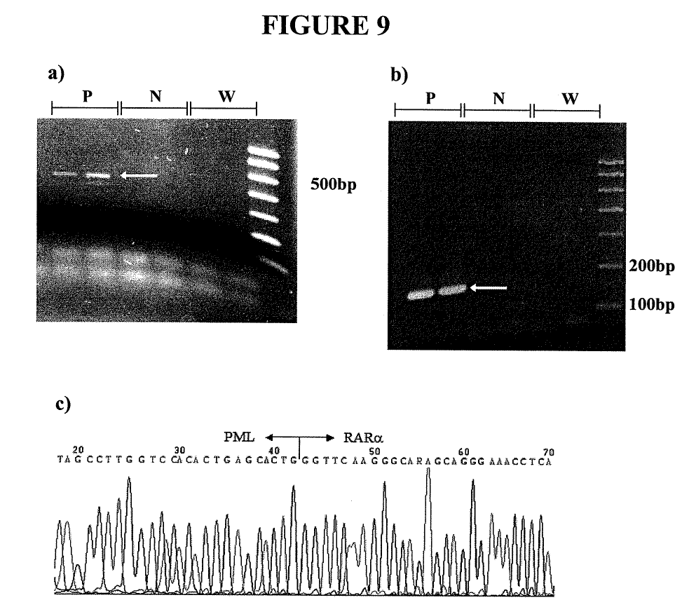

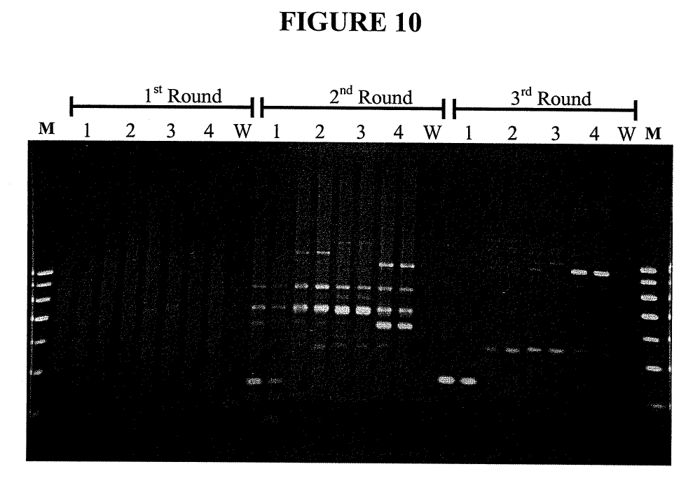

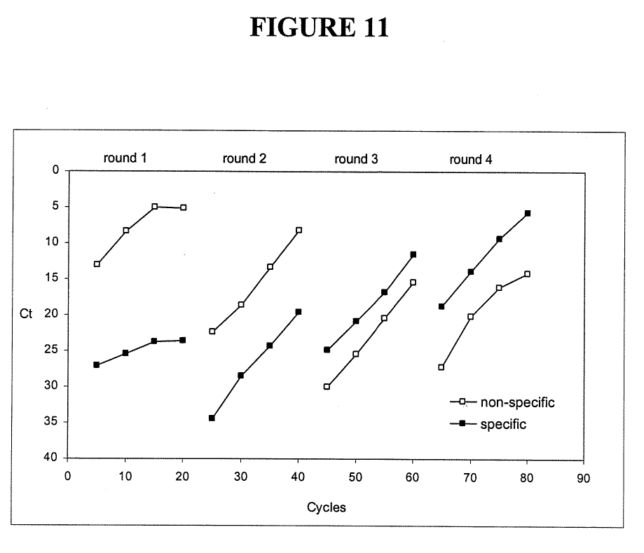

In another example, one may wish to adapt the current method to combine in various ways one or more amplifications using the current method with one or more other amplification steps in order to increase specificity and facilitate isolation of the desired product. FIGS. 6 and 11 illustrate the results of experiments in which 1 standard PCR was followed by 3 rounds of bottleneck PCR. FIG. 7 illustrates the results of an experiment in which 2 rounds of standard PCR were followed by 2 cycles of bottleneck PCR. FIGS. 9, 10 and 12 illustrate the results of experiments in which 1 standard PCR was followed by 2 rounds of bottleneck PCR. It is also possible to alternate cycles of standard and bottleneck PCR. Accordingly, one can optimise the amplification of the sequences of interest by any suitable means such as increasing the number of PCR cycles in the second phase of the method or by performing a subsequent PCR or other form of amplification. The method can also be applied repeatedly in order to provide further enrichment and amplification of the sequence of interest, to the degree desired. Examples showing progressive enrichment of a sequence of interest by repeated application of the method are shown in FIGS. 5 and 11; calculation using the data suggest that the efficiency of the inefficient primers used in rounds 2 and 3 of these experiments was approximately 1% of maximum. These various experimental designs will be understood by those of skill in the art.

The provision of an efficient means of amplifying a nucleic acid region of interest is useful in a range of applications including, but not limited to, the diagnosis and/or monitoring of disease conditions which are characterised by specific gene sequences, the characterisation or analysis of gene regions of interest, the identification or characterisation of DNA breakpoint regions and the isolation of gene sequences of interest where only the nucleotide sequence at one end of the gene sequence of interest is either known or can be inferred.

Yet another aspect of the present invention is directed to a method of amplifying a DNA region of interest, said method comprising:
(i) contacting a DNA sample with:
   (a) one or more forward primers directed to said region of interest; and
   (b) one or more reverse primers directed to said region of interest
   wherein the primers of either group (a) or group (b) hybridise inefficiently and are both functionally inefficient and are operably linked at their 5′ end to an oligonucleotide tag;
(ii) amplifying the DNA sample of step (i) through at least two cycles of amplification;
(iii) contacting the amplified DNA of step (ii) with a primer which is directed to part or all of the sequence which is complementary to that of the oligonucleotide tag of step (i);
(iv) amplifying the DNA sample of step (iii); and
(v) isolating and/or analysing said amplified DNA.

The present invention is further described by reference to the following non-limiting examples.

### Example 1

### BCR-ABL DNA Breakpoint Amplification Using Bottleneck PCR

BCR ABL DNA breakpoints were amplified using BCR and ABL-specific primers in a four round PCR screen. Six BCR-specific primers and 282 ABL-specific primers were designed spanning the major breakpoint regions of BCR (3.2 kb) and ABL (140 kb) DNA respectively.

The first round PCR amplifications were performed in 25 μls containing 50 ng of a single BCR-specific primer, 100 ng of all 282ABL-specific primers (350 pg of each primer), 50 ng of Tag A, 50 ng genomic DNA, 50 mM KCl, 2 mM Tris HCl (pH 8.4), 1 U Platinum Taq DNA polymerase (Invitrogen), 5 mM MgCl₂ and 300 μm of each of dUTP, dATP, dGTP and dTTP. The amplification conditions were: 95° for 4 minutes; then 6 cycles with 97° for 1 minute, 65° for 1 minute with the temperature decreasing 1° every 2 cycles, 72° for 1 minute; then 4 cycles with 96° for 30 seconds, 62° for 1 minute with the temperature decreasing 1° after the first 2 cycles, 72° for 1 minute; then 10 cycles with 94° for 30 secs, 61° for 1 minute, 72° for 1 minute.

The ABL-specific primers have a Tag region (Tag A) at the 5′ end of the primer that is not complementary to ABL DNA. In the first round of PCR the Tag sequence attached to the ABL-specific primers is incorporated into amplicons, enabling the DNA to be further amplified in subsequent rounds of PCR using the BCR primer together with the Tag A primer rather than the ABL-specific reverse primers. Each round of PCR uses different Tag sequences.

The second, third and fourth round PCR amplifications were performed in 25 μls containing a dilution of the previous PCR round reaction mix (at a dilution factor of 100), 50 ng of a single BCR-specific nested primer, 500 pg chimaeric Tag primers (Tag A/I, I/I, 1/2 in the second, third and fourth rounds respectively), 50 ng of a single Tag primer (Tag I, 1, 2 in the second, third and fourth rounds respectively), 50 mM KCl, 2 mM Tris HCl (pH 8.4), 1 U Platinum Taq DNA polymerase (Invitrogen), 5 mM MgCl₂ and 300 μm of each of dUTP, dATP, dGTP and dTTP. The amplification conditions were: 95° for 4 minutes; then 20 cycles with 94° for 30 seconds, 65° for 1 minute, 72° for 1 minute.

### Example 2

### PML-RARα DNA Breakpoint Amplification Using Bottleneck PCR

Bottleneck PCR was used to isolate the PML-RARα translocation breakpoint from a patient with acute promyelocytic leukemia. The patient DNA was amplified using multiple RARα primers and a single PML primer and then 2 rounds of bottleneck PCR were performed. The amplified DNA electrophoresed on a 2% agarose gel (FIG. 9a). To confirm that the breakpoint had been isolated, the breakpoint sequence was used to design RARα and PML primers spanning the breakpoint, and the patient DNA was amplified for one round using these primers (FIG. 9b). The sequence of the amplified band shown on the gel in FIG. 9a was also determined (FIG. 9c).

### Example 3

### Gene Walking Using a Degenerate Primer and Bottleneck PCR

Gene walking along three genes, APC, BRCA1 and myocillin, was performed using 50 ng of a gene-specific forward primer, 50 ng of one of a variety of degenerate reverse primers, and 50 ng of a reverse tag primer. The degenerate reverse primers had 4-6 random normal residues at the 3′ end, followed by 3-6 degenerate residues, followed by a random tag sequence of 12-18, usually 18, normal residues. The most commonly used degenerate primer had 5 fixed bases at the 3′ end followed by 5 degenerate bases, followed by a tag sequence of 18 bases (5′TGCTAGGATCCAAGGNNNNNATTCG3′ (SEQ ID NO:1)). The reverse tag primer had the same sequence as the tag on the degenerate reverse primer. Five PCR cycles, with annealing at 35° C. for five minutes, were followed by 15 cycles with annealing at 55° C. for 3 minutes.

PCR was performed in a 25 μl volume, with 50 ng total DNA, 5 mM MgCl₂, 0.1 mM dUTP, 0.2 mM dTTP, and 0.3 mM of each of dCTP, dATP and dGTP, and 1 unit of Platinum Taq polymerase (Invitrogen). Primers were from Sigma-Aldrich (St. Louis, Mo., USA) or Invitrogen (Carlsbad, Calif., USA). PCR cycling conditions typically involved denaturation at 94° C. for 30 seconds, annealing as described above, and extension at 72° C. for 90 seconds.

Between 1 and 3 rounds of bottleneck PCR were performed, usually 2. A 1/100 dilution of the amplified material from the primary round described above was amplified in the first bottleneck PCR and a 1/1000 dilution of the amplified material from the previous round was used for each subsequent round. Each PCR round was run for 20 cycles except when electrophoresis was to be performed, in which case it was run to completion, for 30-40 cycles. Each PCR contained 50 ng of a nested forward primer, 0.5 ng of a hybrid reverse primer and 50 ng of a tag reverse primer. The hybrid reverse primer consisted of a 3′ end, which had the same sequence as the tag primer for the previous PCR round, and a 5′ end, which had a new tag sequence. The tag reverse primer had the same sequence as the new tag sequence.

PCR products were examined by gel electrophoresis and discrete bands were isolated and sequenced usually with the forward primer and the tag primer used for the final amplification. In some experiments involving gene walking, the entire amplified product was sequenced, irrespective of whether 1 or more discrete bands had been visualised. In one experiment involving gene walking, the sequencing reaction was performed using primers 732 by and 1302 by downstream from the initial sequencing primer.

### Example 4

FIG. 13 provides examples of primers and tag sequences suitable for use in the method of the present invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Cheng et al., 1994, Effective amplification of long targets from cloned inserts and human genomic DNA. Proc Natl Acad Sci. 91:5695-5699
Sambrook and Russel, 2001, Molecular Cloning: A Laboratory Manual, 3^{rd} Ed. Cold Spring Harbor, N.W.: Cold Spring Harbor Laboratory Press. Chapter 8: In vitro Amplification of DNA by the Polymerase Chain Reaction.
Nailis H, Coenye T, Van Nieuwerburgh F, Deforce D, Nelis H J (2006). “Development and evaluation of different normalization strategies for gene expression studies in Candida albicans biofilms by real-time PCR”. BMC Mol Biol. 7: 25
Nolan T, Hands R E, Bustin S A (2006). “Quantification of mRNA using real-time RT-PCR”. Nat. Protoc. 1: 1559-1582

1. A method of amplifying a nucleic acid region of interest, said method comprising:
(i) contacting a nucleic acid sample with:
(a) one or more forward primers directed to said region of interest; and
(b) one or more reverse primers directed to said region of interest
wherein the primers of either group (a) or group (b) are functionally inefficient and are operably linked at their 5′ end to an oligonucleotide tag;
(ii) amplifying the nucleic acid sample of step (i) through at least two cycles of amplification;
(iii) contacting the amplified nucleic acid of step (ii) with a primer which is directed to part or all of the sequence which is complementary to that of the oligonucleotide tag of step (i); and
(iv) amplifying the nucleic acid sample of step (iii).

2. The method according to claim 1 wherein said nucleic acid is DNA.

3. The method according to claim 1, wherein said amplified nucleic acid of step (iv) is isolated or analysed.

4. The method of claim 1, wherein said nucleic acid region of interest is a region of a DNA molecule, which has been generated by an amplification method.

5. The method according to claim 4 wherein said amplification method is PCR.

6. The method of claim 1, wherein the amplification methods of steps (ii) and (iv) are PCR, NASBA or strand displacement amplification.

7. The method of claim 1, wherein said primer is 4-60 nucleotides in length, 10-50 nucleotides in length, 15-45 nucleotides in length, 20-40 nucleotides in length or 25-35 nucleotides in length.

8. The method according to claim 7 wherein said primer is 26, 27, 28, 29, 30, 31, 32, 33 or 34 nucleotides in length.

9. The method of claim 1, wherein said primer is rendered functionally inefficient by:
(i) reducing the concentration of primer which is used;
(ii) reducing the hybridisation time of the primer;
(iii) increasing the temperature at which the hybridisation reaction is required to occur;
(iv) modifying the length of the primer;
(v) altering the primer melting temperature;
(vi) introducing a chaotropic agent during the hybridisation or primer extension phase;
(vii) introducing into the reaction a competitive inhibitor to the primer;
(viii) introducing nucleotide mismatches into the primer sequence; and
(ix) providing primer analogs which hydrogen-bond less efficiently in the context of hybridisation.

10. The method of claim 1, wherein the primer group, which is rendered inefficient is the primer group, can hybridise promiscuously to non-target DNA regions.

11. The method according to claim 10 wherein said primer group is a degenerate primer.

12. The method of claim 1, wherein said region of interest is a region of genomic DNA, a gene, part of a gene, a DNA recombination product or a chromosomal gene translocation breakpoint.

13. The method according to claim 12 wherein said chromosomal gene translocation breakpoint is BCR-ABL.

14. The method according to claim 12 wherein said chromosomal gene translocation breakpoint is PML-RARα.

15. The method according to claim 13 wherein there is used 1-20 forward primers directed to BCR and 250-350 reverse primers directed to ABL.

16. The method according to claim 15 wherein there is used 12 forward primers directed to BCR and 282 reverse primers directed to ABL.

17. The method according to claim 15 wherein there is used 6 forward primers directed to BCR and 24 reverse primers directed to ABL.

18. The method according to claim 15 wherein there is used 1 forward primer directed to BCR and 282 reverse primers directed to ABL.

19. The method according to claim 15 wherein there is used 6 forward primers directed to BCR and 270-310 reverse primers directed to ABL.


SEQUENCE LISTING (398 sequences)

SEQ ID NO: 1 - DNA, 25 bp, Artificial Sequence
  misc_feature (16)..(20): n is a, c, g, or t
tgctaggatccaaggnnnnnattcg

SEQ ID NO: 2 - DNA, 53 bp, Artificial Sequence
  Synthetic
tagccttggtccacactgagcactgggttcaagggcaragcagggaaacctca

SEQ ID NO: 3 - DNA, 52 bp, Artificial Sequence
  Synthetic
tcagcacttatcagaaacttttgacaatatagacaatttaagtcccaaggca

SEQ ID NO: 4 - DNA, 52 bp, Artificial Sequence
  Synthetic
tacccagccgamctagcccataaaatacatagtgcaaatcwtatggatgata

SEQ ID NO: 5 - DNA, 52 bp, Artificial Sequence
  Synthetic
cacagaaacagtcattttcattctcaaagagttcatctggacaaagcagtaa

SEQ ID NO: 6 - DNA, 23 bp, Artificial Sequence
  Synthetic
gcaacactgtgacgtactggagg

SEQ ID NO: 7 - DNA, 23 bp, Artificial Sequence
  Synthetic
gcaacactgtgacgtactggagg

SEQ ID NO: 8 - DNA, 46 bp, Artificial Sequence
  Synthetic
tcgcttctagtctgtgccacagggcaacactgtgacgtactggagg

SEQ ID NO: 9 - DNA, 23 bp, Artificial Sequence
  Synthetic
tcgcttctagtctgtgccacagg

SEQ ID NO: 10 - DNA, 45 bp, Artificial Sequence
  Synthetic
gacatgtgctggagatatgacgtcgcttctagtctgtgccacagg

SEQ ID NO: 11 - DNA, 22 bp, Artificial Sequence
  Synthetic
gacatgtgctggagatatgacg

SEQ ID NO: 12 - DNA, 44 bp, Artificial Sequence
  Synthetic
gagaaggtgatgcaacatacgggacatgtgctggagatatgacg

SEQ ID NO: 13 - DNA, 22 bp, Artificial Sequence
  Synthetic
gagaaggtgatgcaacatacgg

SEQ ID NO: 14 - DNA, 22 bp, Artificial Sequence
  Synthetic
gcagtacaaacaacgcacagcg

SEQ ID NO: 15 - DNA, 22 bp, Artificial Sequence
  Synthetic
gcagtacaaacaacgcacagcg

SEQ ID NO: 16 - DNA, 43 bp, Artificial Sequence
  Synthetic
gccatctacattgccctgtccgcagtacaaacaacgcacagcg

SEQ ID NO: 17 - DNA, 21 bp, Artificial Sequence
  Synthetic
gccatctacattgccctgtcc

SEQ ID NO: 18 - DNA, 41 bp, Artificial Sequence
  Synthetic
gttgagacgtggcaggaacggccatctacattgccctgtcc

SEQ ID NO: 19 - DNA, 20 bp, Artificial Sequence
  Synthetic
gttgagacgtggcaggaacg

SEQ ID NO: 20 - DNA, 44 bp, Artificial Sequence
  Synthetic
catgcgattattgcactctcttccgttgagacgtggcaggaacg

SEQ ID NO: 21 - DNA, 24 bp, Artificial Sequence
  Synthetic
catgcgattattgcactctcttcc

SEQ ID NO: 22 - DNA, 25 bp, Artificial Sequence
  misc_feature (16)..(20): n is a, c, g, or t
tgctaggatccaaggnnnnnattcg

SEQ ID NO: 23 - DNA, 18 bp, Artificial Sequence
  Synthetic
tcatgctaggatccaagg

SEQ ID NO: 24 - DNA, 40 bp, Artificial Sequence
  Synthetic
gcagttcatggtcccgtcatcctcatgctaggatccaagg

SEQ ID NO: 25 - DNA, 22 bp, Artificial Sequence
  Synthetic
gcagttcatggtcccgtcatcc

SEQ ID NO: 26 - DNA, 44 bp, Artificial Sequence
  Synthetic
aaagaagagtcgcagaggctcggcagttcatggtcccgtcatcc

SEQ ID NO: 27 - DNA, 22 bp, Artificial Sequence
  Synthetic
aaagaagagtcgcagaggctcg

SEQ ID NO: 28 - DNA, 44 bp, Artificial Sequence
  Synthetic
tccttccttggcaacgacatcgaaagaagagtcgcagaggctcg

SEQ ID NO: 29 - DNA, 22 bp, Artificial Sequence
  Synthetic
tccttccttggcaacgacatcg

SEQ ID NO: 30 - DNA, 20 bp, Artificial Sequence
  Synthetic
cttctccctgacatccgtgg

SEQ ID NO: 31 - DNA, 24 bp, Artificial Sequence
  Synthetic
tgacatccgtggagctgcagatgc

SEQ ID NO: 32 - DNA, 47 bp, Artificial Sequence
  Synthetic
tggcacgtgatcgtagagtgagggagctgcagatgctgaccaactcg

SEQ ID NO: 33 - DNA, 46 bp, Artificial Sequence
  Synthetic
cggcatccttgtcactgactgctgaccaactcgtgtgtgaaactcc

SEQ ID NO: 34 - DNA, 21 bp, Artificial Sequence
  Synthetic
ccagccctcctctcctccagc

SEQ ID NO: 35 - DNA, 23 bp, Artificial Sequence
  Synthetic
ctctcctccagctacctgccagc

SEQ ID NO: 36 - DNA, 45 bp, Artificial Sequence
  Synthetic
tggcacgtgatcgtagagtgaggcagccggcacttttggtcaagc

SEQ ID NO: 37 - DNA, 47 bp, Artificial Sequence
  Synthetic
cggcatccttgtcactgactgcggcacttttggtcaagctgttttgc

SEQ ID NO: 38 - DNA, 22 bp, Artificial Sequence
  Synthetic
gaggttgttcagatgaccacgg

SEQ ID NO: 39 - DNA, 24 bp, Artificial Sequence
  Synthetic
accacgggacacctttgaccctgg

SEQ ID NO: 40 - DNA, 47 bp, Artificial Sequence
  Synthetic
tggcacgtgatcgtagagtgaggcacctttgaccctggccgctgtgg

SEQ ID NO: 41 - DNA, 46 bp, Artificial Sequence
  Synthetic
cggcatccttgtcactgactgcctggccgctgtggagtgtttgtgc

SEQ ID NO: 42 - DNA, 24 bp, Artificial Sequence
  Synthetic
cagctactggagctgtcagaacag

SEQ ID NO: 43 - DNA, 24 bp, Artificial Sequence
  Synthetic
ctggagctgtcagaacagtgaagg

SEQ ID NO: 44 - DNA, 47 bp, Artificial Sequence
  Synthetic
tggcacgtgatcgtagagtgaggcagtgaaggctggtaacacatgag

SEQ ID NO: 45 - DNA, 46 bp, Artificial Sequence
  Synthetic
cggcatccttgtcactgactgctggtaacacatgagttgcactgtg

SEQ ID NO: 46 - DNA, 17 bp, Artificial Sequence
  Synthetic
tgggcctccctgcatcc

SEQ ID NO: 47 - DNA, 24 bp, Artificial Sequence
  Synthetic
tccctgcatccctgcatctcctcc

SEQ ID NO: 48 - DNA, 47 bp, Artificial Sequence
  Synthetic
tggcacgtgatcgtagagtgaggatccctgcatctcctcccgggtcc

SEQ ID NO: 49 - DNA, 46 bp, Artificial Sequence
  Synthetic
cggcatccttgtcactgactgctcctcccgggtcctgtctgtgagc

SEQ ID NO: 50 - DNA, 17 bp, Artificial Sequence
  Synthetic
tccccctgcaccccacg

SEQ ID NO: 51 - DNA, 24 bp, Artificial Sequence
  Synthetic
cccacgacttctccagcactgagc

SEQ ID NO: 52 - DNA, 47 bp, Artificial Sequence
  Synthetic
tggcacgtgatcgtagagtgaggccagcactgagctgcttcctgtgc

SEQ ID NO: 53 - DNA, 46 bp, Artificial Sequence
  Synthetic
cggcatccttgtcactgactgcgctgcttcctgtgccccacagtgg

SEQ ID NO: 54 - DNA, 23 bp, Artificial Sequence
  Synthetic
tggcacgtgatcgtagagtgagg

SEQ ID NO: 55 - DNA, 22 bp, Artificial Sequence
  Synthetic
cggcatccttgtcactgactgc

SEQ ID NO: 56 - DNA, 49 bp, Artificial Sequence
  Synthetic
gcaacactgtgacgtactggagggtctatctaaaattcacaaggaatgc

SEQ ID NO: 57 - DNA, 47 bp, Artificial Sequence
  Synthetic
gcaacactgtgacgtactggaggaggcaaagtaaaatccaagcaccc

SEQ ID NO: 58 - DNA, 44 bp, Artificial Sequence
  Synthetic
gcaacactgtgacgtactggaggcactcctgcactccagcctgg

SEQ ID NO: 59 - DNA, 47 bp, Artificial Sequence
  Synthetic
gcaacactgtgacgtactggaggcaaccaccaaagtgcttttcctgg

SEQ ID NO: 60 - DNA, 49 bp, Artificial Sequence
  Synthetic
gcaacactgtgacgtactggaggatatggcatctgtaaatattaccacc

SEQ ID NO: 61 - DNA, 44 bp, Artificial Sequence
  Synthetic
gcaacactgtgacgtactggaggtgcctcggcctcccaaagtgc

SEQ ID NO: 62 - DNA, 43 bp, Artificial Sequence
  Synthetic
gcaacactgtgacgtactggaggagccaccacacccagccagg

SEQ ID NO: 63 - DNA, 48 bp, Artificial Sequence
  Synthetic
gcaacactgtgacgtactggaggaataactgttttctccccccaaaac

SEQ ID NO: 64 - DNA, 48 bp, Artificial Sequence
  Synthetic
gcaacactgtgacgtactggaggtgttttacaaaaatggggccatacc

SEQ ID NO: 65 - DNA, 51 bp, Artificial Sequence
  Synthetic
gcaacactgtgacgtactggaggacttaagcaaattctttcataaaaaggg

SEQ ID NO: 66 - DNA, 48 bp, Artificial Sequence
  Synthetic
gcaacactgtgacgtactggaggctttcaattgttgtaccaactctcc

SEQ ID NO: 67 - DNA, 46 bp, Artificial Sequence
  Synthetic
gcaacactgtgacgtactggaggacctcctgcatctctccttttgc

SEQ ID NO: 68 - DNA, 50 bp, Artificial Sequence
  Synthetic
gcaacactgtgacgtactggaggaaataaagttttgagaaccataagtgg

SEQ ID NO: 69 - DNA, 45 bp, Artificial Sequence
  Synthetic
gcaacactgtgacgtactggaggcaccatcacagctcactgcagc

SEQ ID NO: 70 - DNA, 47 bp, Artificial Sequence
  Synthetic
gcaacactgtgacgtactggaggaacctctttgagaatcggatagcc

SEQ ID NO: 71 - DNA, 50 bp, Artificial Sequence
  Synthetic
gcaacactgtgacgtactggaggaaataaagtacatacctccaattttgc

SEQ ID NO: 72 - DNA, 48 bp, Artificial Sequence
  Synthetic
gcaacactgtgacgtactggagggacacattcctatgggtttaattcc

SEQ ID NO: 73 - DNA, 49 bp, Artificial Sequence
  Synthetic
gcaacactgtgacgtactggaggtgtaaaatatggtttcagaagggagg

SEQ ID NO: 74 - DNA, 45 bp, Artificial Sequence
  Synthetic
gcaacactgtgacgtactggagggcaggtggataacgaggtcagg

SEQ ID NO: 75 - DNA, 48 bp, Artificial Sequence
  Synthetic
gcaacactgtgacgtactggaggccagccaagaatttcaaagattagc

SEQ ID NO: 76 - DNA, 46 bp, Artificial Sequence
  Synthetic
gcaacactgtgacgtactggagggaagggagatgacaaagggaacg

SEQ ID NO: 77 - DNA, 46 bp, Artificial Sequence
  Synthetic
gcaacactgtgacgtactggagggcagaagaactgcttgaacctgg

SEQ ID NO: 78 - DNA, 45 bp, Artificial Sequence
  Synthetic
gcaacactgtgacgtactggagggtggtcccagctactcgagagg

SEQ ID NO: 79 - DNA, 48 bp, Artificial Sequence
  Synthetic
gcaacactgtgacgtactggaggccctcagcaaaactaactgaaaagg

SEQ ID NO: 80 - DNA, 49 bp, Artificial Sequence
  Synthetic
gcaacactgtgacgtactggaggtagaaaccaagatatctagaattccc

SEQ ID NO: 81 - DNA, 45 bp, Artificial Sequence
  Synthetic
gcaacactgtgacgtactggaggccacgcccggcggaataaatgc

SEQ ID NO: 82 - DNA, 49 bp, Artificial Sequence
  Synthetic
gcaacactgtgacgtactggaggacaaaaaaagaggcaaaaactgagag

SEQ ID NO: 83 - DNA, 44 bp, Artificial Sequence
  Synthetic
gcaacactgtgacgtactggaggctgggcgcagtggctcatgcc

SEQ ID NO: 84 - DNA, 45 bp, Artificial Sequence
  Synthetic
gcaacactgtgacgtactggaggtggctgtgaggctgagaactgc

SEQ ID NO: 85 - DNA, 45 bp, Artificial Sequence
  Synthetic
gcaacactgtgacgtactggaggctgggcgacagagtgagactcc

SEQ ID NO: 86 - DNA, 44 bp, Artificial Sequence
  Synthetic
gcaacactgtgacgtactggaggaagtctggctgggcgcagtgg

SEQ ID NO: 87 - DNA, 47 bp, Artificial Sequence
  Synthetic
gcaacactgtgacgtactggaggaatggacaaaagaggtgaactggc

SEQ ID NO: 88 - DNA, 47 bp, Artificial Sequence
  Synthetic
gcaacactgtgacgtactggagggatagagtgaaaacgcacaatggc

SEQ ID NO: 89 - DNA, 49 bp, Artificial Sequence
  Synthetic
gcaacactgtgacgtactggaggaattaaacagctaggtcaatatgagg

SEQ ID NO: 90 - DNA, 46 bp, Artificial Sequence
  Synthetic
gcaacactgtgacgtactggaggggtctccactatcaagggacaag

SEQ ID NO: 91 - DNA, 47 bp, Artificial Sequence
  Synthetic
gcaacactgtgacgtactggaggaagcagctgttagtcatttccagg

SEQ ID NO: 92 - DNA, 46 bp, Artificial Sequence
  Synthetic
gcaacactgtgacgtactggaggaggcatcctcagattatggctcc

SEQ ID NO: 93 - DNA, 46 bp, Artificial Sequence
  Synthetic
gcaacactgtgacgtactggaggcctgagtaacactgagaccctgc

SEQ ID NO: 94 - DNA, 47 bp, Artificial Sequence
  Synthetic
gcaacactgtgacgtactggaggaacactcaagctgtcaagagacac

SEQ ID NO: 95 - DNA, 44 bp, Artificial Sequence
  Synthetic
gcaacactgtgacgtactggaggattcaggccaggcgcagtggc

SEQ ID NO: 96 - DNA, 47 bp, Artificial Sequence
  Synthetic
gcaacactgtgacgtactggaggtaaatcgtaaaactgccacaaagc

SEQ ID NO: 97 - DNA, 47 bp, Artificial Sequence
  Synthetic
gcaacactgtgacgtactggaggcagaggagtaggagaaggaaaagg

SEQ ID NO: 98 - DNA, 48 bp, Artificial Sequence
  Synthetic
gcaacactgtgacgtactggaggggtagctatctaccaagtagaatcc

SEQ ID NO: 99 - DNA, 48 bp, Artificial Sequence
  Synthetic
gcaacactgtgacgtactggaggatcagattggaaaaagtcccaaagc

SEQ ID NO: 100 - DNA, 46 bp, Artificial Sequence
  Synthetic
gcaacactgtgacgtactggaggctcctgaaaagcacctactcagc

SEQ ID NO: 101 - DNA, 46 bp, Artificial Sequence
  Synthetic
gcaacactgtgacgtactggaggctccttaaacctgaggtactggg

SEQ ID NO: 102 - DNA, 48 bp, Artificial Sequence
  Synthetic
gcaacactgtgacgtactggaggttttctcctaatagaccaccattcc

SEQ ID NO: 103 - DNA, 48 bp, Artificial Sequence
  Synthetic
gcaacactgtgacgtactggaggctgctgtattaccatcactcatgtc

SEQ ID NO: 104 - DNA, 46 bp, Artificial Sequence
  Synthetic
gcaacactgtgacgtactggaggctggccaacatagtgaaaccacg

SEQ ID NO: 105 - DNA, 50 bp, Artificial Sequence
  Synthetic
gcaacactgtgacgtactggaggatttgaataggggttaaagtatcattg

SEQ ID NO: 106 - DNA, 46 bp, Artificial Sequence
  Synthetic
gcaacactgtgacgtactggaggcacttcagtggaagttggcatgc

SEQ ID NO: 107 - DNA, 50 bp, Artificial Sequence
  Synthetic
gcaacactgtgacgtactggagggtttttcttcgaagtgataaacatacg

SEQ ID NO: 108 - DNA, 47 bp, Artificial Sequence
  Synthetic
gcaacactgtgacgtactggagggctccttagtctatgtacctgtgg

SEQ ID NO: 109 - DNA, 46 bp, Artificial Sequence
  Synthetic
gcaacactgtgacgtactggaggtactctggcatggtaactggtgc

SEQ ID NO: 110 - DNA, 46 bp, Artificial Sequence
  Synthetic
gcaacactgtgacgtactggaggacaaaggactaggtctgtggagc

SEQ ID NO: 111 - DNA, 51 bp, Artificial Sequence
  Synthetic
gcaacactgtgacgtactggaggccaagtttaccaaattaccaaagttacc

SEQ ID NO: 112 - DNA, 45 bp, Artificial Sequence
  Synthetic
gcaacactgtgacgtactggaggtgagccgatatcacgccactgc

SEQ ID NO: 113 - DNA, 47 bp, Artificial Sequence
  Synthetic
gcaacactgtgacgtactggaggtcccaataaaggttttggcccagg

SEQ ID NO: 114 - DNA, 47 bp, Artificial Sequence
  Synthetic
gcaacactgtgacgtactggaggctgggtagcaaattagggaacagg

SEQ ID NO: 115 - DNA, 48 bp, Artificial Sequence
  Synthetic
gcaacactgtgacgtactggaggctggccagaaaagacagttttatcc

SEQ ID NO: 116 - DNA, 46 bp, Artificial Sequence
  Synthetic
gcaacactgtgacgtactggaggggttcccaggaagggataacacc

SEQ ID NO: 117 - DNA, 46 bp, Artificial Sequence
  Synthetic
gcaacactgtgacgtactggaggtcactccaggaggttccatttcc

SEQ ID NO: 118 - DNA, 47 bp, Artificial Sequence
  Synthetic
gcaacactgtgacgtactggaggaggcttggaaataagcagcagtgg

SEQ ID NO: 119 - DNA, 49 bp, Artificial Sequence
  Synthetic
gcaacactgtgacgtactggaggattcatacaatggaatactactcagc

SEQ ID NO: 120 - DNA, 46 bp, Artificial Sequence
  Synthetic
gcaacactgtgacgtactggaggtaagtgatcctcccacctcaacc

SEQ ID NO: 121 - DNA, 46 bp, Artificial Sequence
  Synthetic
gcaacactgtgacgtactggaggtataagaggaagactggggctgg

SEQ ID NO: 122 - DNA, 48 bp, Artificial Sequence
  Synthetic
gcaacactgtgacgtactggaggtcatacttatgcaggttataggagg

SEQ ID NO: 123 - DNA, 45 bp, Artificial Sequence
  Synthetic
gcaacactgtgacgtactggaggcaagatcacgccactgcactcc

SEQ ID NO: 124 - DNA, 49 bp, Artificial Sequence
  Synthetic
gcaacactgtgacgtactggaggaaaataaatagctggtgctcaagatc

SEQ ID NO: 125 - DNA, 46 bp, Artificial Sequence
  Synthetic
gcaacactgtgacgtactggaggcaccagcctcattcaacagatgg

SEQ ID NO: 126 - DNA, 45 bp, Artificial Sequence
  Synthetic
gcaacactgtgacgtactggaggcaatgcagcctcaacctcctgg

SEQ ID NO: 127 - DNA, 46 bp, Artificial Sequence
  Synthetic
gcaacactgtgacgtactggagggttaggtcaggtgctcatgtctg

SEQ ID NO: 128 - DNA, 50 bp, Artificial Sequence
  Synthetic
gcaacactgtgacgtactggaggaagtttcaaaaggacatgtacaaaatg

SEQ ID NO: 129 - DNA, 45 bp, Artificial Sequence
  Synthetic
gcaacactgtgacgtactggaggtcctgaagaggctgcagcttcc

SEQ ID NO: 130 - DNA, 46 bp, Artificial Sequence
  Synthetic
gcaacactgtgacgtactggaggctggtgcacattcccaagtgtgc

SEQ ID NO: 131 - DNA, 47 bp, Artificial Sequence
  Synthetic
gcaacactgtgacgtactggaggcatgttggccatgttcttctgagg

SEQ ID NO: 132 - DNA, 44 bp, Artificial Sequence
  Synthetic
gcaacactgtgacgtactggaggctcagcctcccgagtagctgg

SEQ ID NO: 133 - DNA, 48 bp, Artificial Sequence
  Synthetic
gcaacactgtgacgtactggaggaaagacatttaagaggagatgaggc

SEQ ID NO: 134 - DNA, 45 bp, Artificial Sequence
  Synthetic
gcaacactgtgacgtactggaggtgctgggattacaggcgtgagc

SEQ ID NO: 135 - DNA, 45 bp, Artificial Sequence
  Synthetic
gcaacactgtgacgtactggaggtgtgacttccatccgcagctcc

SEQ ID NO: 136 - DNA, 47 bp, Artificial Sequence
  Synthetic
gcaacactgtgacgtactggagggacacttttgtggagctttcatgg

SEQ ID NO: 137 - DNA, 45 bp, Artificial Sequence
  Synthetic
gcaacactgtgacgtactggaggcatgtgagggggcacgtcttgc

SEQ ID NO: 138 - DNA, 48 bp, Artificial Sequence
  Synthetic
gcaacactgtgacgtactggaggtcttctctatgagaaaagtggttgc

SEQ ID NO: 139 - DNA, 46 bp, Artificial Sequence
  Synthetic
gcaacactgtgacgtactggaggtggcaaaatgctatcgagctgcc

SEQ ID NO: 140 - DNA, 45 bp, Artificial Sequence
  Synthetic
gcaacactgtgacgtactggaggtatgaacacagccggcctcagg

SEQ ID NO: 141 - DNA, 46 bp, Artificial Sequence
  Synthetic
gcaacactgtgacgtactggagggaggttgcagtgagctgagatcg

SEQ ID NO: 142 - DNA, 45 bp, Artificial Sequence
  Synthetic
gcaacactgtgacgtactggagggtcaagcacccagtccgatacc

SEQ ID NO: 143 - DNA, 44 bp, Artificial Sequence
  Synthetic
gcaacactgtgacgtactggaggatctgggcttggtggcgcacg

SEQ ID NO: 144 - DNA, 45 bp, Artificial Sequence
  Synthetic
gcaacactgtgacgtactggagggttaagcgggtcccacatcagc

SEQ ID NO: 145 - DNA, 47 bp, Artificial Sequence
  Synthetic
gcaacactgtgacgtactggaggcagccagtttcagtagaaagatgc

SEQ ID NO: 146 - DNA, 46 bp, Artificial Sequence
  Synthetic
gcaacactgtgacgtactggagggacccaagcataaggggactagc

SEQ ID NO: 147 - DNA, 50 bp, Artificial Sequence
  Synthetic
gcaacactgtgacgtactggaggcccaaaaagtttacaagagaaattttc

SEQ ID NO: 148 - DNA, 45 bp, Artificial Sequence
  Synthetic
gcaacactgtgacgtactggaggcgcctgtagtcccagctactcg

SEQ ID NO: 149 - DNA, 46 bp, Artificial Sequence
  Synthetic
gcaacactgtgacgtactggaggcgcgtgatgcggaaaagaaatcc

SEQ ID NO: 150 - DNA, 47 bp, Artificial Sequence
  Synthetic
gcaacactgtgacgtactggaggtctactatgaaccctccttcagac

SEQ ID NO: 151 - DNA, 46 bp, Artificial Sequence
  Synthetic
gcaacactgtgacgtactggagggtgctgggattacaggtgtgagc

SEQ ID NO: 152 - DNA, 46 bp, Artificial Sequence
  Synthetic
gcaacactgtgacgtactggaggttatccaaatgtcccagggcagg

SEQ ID NO: 153 - DNA, 44 bp, Artificial Sequence
  Synthetic
gcaacactgtgacgtactggaggctgccagcactgctcgccagc

SEQ ID NO: 154 - DNA, 45 bp, Artificial Sequence
  Synthetic
gcaacactgtgacgtactggagggctactgcaggcagtgccttcc

SEQ ID NO: 155 - DNA, 45 bp, Artificial Sequence
  Synthetic
gcaacactgtgacgtactggaggcatccaagcccaaggtgtcagg

SEQ ID NO: 156 - DNA, 48 bp, Artificial Sequence
  Synthetic
gcaacactgtgacgtactggaggtgtttgcatgtaatttcaggaagcc

SEQ ID NO: 157 - DNA, 47 bp, Artificial Sequence
  Synthetic
gcaacactgtgacgtactggagggatccgtcactgttaacactcagg

SEQ ID NO: 158 - DNA, 46 bp, Artificial Sequence
  Synthetic
gcaacactgtgacgtactggaggctcacagtcacaagctcctgagc

SEQ ID NO: 159 - DNA, 45 bp, Artificial Sequence
  Synthetic
gcaacactgtgacgtactggagggagatgatgctggggtcacagg

SEQ ID NO: 160 - DNA, 46 bp, Artificial Sequence
  Synthetic
gcaacactgtgacgtactggaggttagaagaatgggatcgcaaagg

SEQ ID NO: 161 - DNA, 47 bp, Artificial Sequence
  Synthetic
gcaacactgtgacgtactggaggcggtattcaaatatgaggtcaggc

SEQ ID NO: 162 - DNA, 46 bp, Artificial Sequence
  Synthetic
gcaacactgtgacgtactggagggtaaatcctgctgccagtcttcc

SEQ ID NO: 163 - DNA, 45 bp, Artificial Sequence
  Synthetic
gcaacactgtgacgtactggaggacagggtcagacagagccttgg

SEQ ID NO: 164 - DNA, 50 bp, Artificial Sequence
  Synthetic
gcaacactgtgacgtactggaggagttattgatctaactatacaacaagc

SEQ ID NO: 165 - DNA, 44 bp, Artificial Sequence
  Synthetic
gcaacactgtgacgtactggaggaaagactaggggccggggacg

SEQ ID NO: 166 - DNA, 49 bp, Artificial Sequence
  Synthetic
gcaacactgtgacgtactggaggctggtagaaataaagacaacaaagcc

SEQ ID NO: 167 - DNA, 50 bp, Artificial Sequence
  Synthetic
gcaacactgtgacgtactggagggtgccaagtaattaaaagtttgaaacc

SEQ ID NO: 168 - DNA, 45 bp, Artificial Sequence
  Synthetic
gcaacactgtgacgtactggaggggcttttgaagggagcaccacc

SEQ ID NO: 169 - DNA, 50 bp, Artificial Sequence
  Synthetic
gcaacactgtgacgtactggagggaaggataaatacctatgatactttcc

SEQ ID NO: 170 - DNA, 47 bp, Artificial Sequence
  Synthetic
gcaacactgtgacgtactggaggggcagggaaatactgtgcttcaag

SEQ ID NO: 171 - DNA, 47 bp, Artificial Sequence
  Synthetic
gcaacactgtgacgtactggagggtggtgaaattccacctcagtacc

SEQ ID NO: 172 - DNA, 46 bp, Artificial Sequence
  Synthetic
gcaacactgtgacgtactggaggtcccaaagtgctgggattacagg

SEQ ID NO: 173 - DNA, 48 bp, Artificial Sequence
  Synthetic
gcaacactgtgacgtactggagggaaattagcaaacaatgccaagacg

SEQ ID NO: 174 - DNA, 46 bp, Artificial Sequence
  Synthetic
gcaacactgtgacgtactggaggtaagtattggaccgggaaggagg

SEQ ID NO: 175 - DNA, 48 bp, Artificial Sequence
  Synthetic
gcaacactgtgacgtactggaggctatcattttgctcaaagtgtagcc

SEQ ID NO: 176 - DNA, 47 bp, Artificial Sequence
  Synthetic
gcaacactgtgacgtactggaggatttcacaaactacagaggccagg

SEQ ID NO: 177 - DNA, 47 bp, Artificial Sequence
  Synthetic
gcaacactgtgacgtactggaggtagacttctgtctctctatgctgc

SEQ ID NO: 178 - DNA, 46 bp, Artificial Sequence
  Synthetic
gcaacactgtgacgtactggaggtgagtgagctgccatgtgatacc

SEQ ID NO: 179 - DNA, 45 bp, Artificial Sequence
  Synthetic
gcaacactgtgacgtactggaggacttcacaccagcctgtccacc

SEQ ID NO: 180 - DNA, 47 bp, Artificial Sequence
  Synthetic
gcaacactgtgacgtactggaggtaactcatatcctcagagagaccc

SEQ ID NO: 181 - DNA, 45 bp, Artificial Sequence
  Synthetic
gcaacactgtgacgtactggaggagaggttcctcgattcccctgc

SEQ ID NO: 182 - DNA, 46 bp, Artificial Sequence
  Synthetic
gcaacactgtgacgtactggagggtgtcagcgtcccaacacaaagc

SEQ ID NO: 183 - DNA, 46 bp, Artificial Sequence
  Synthetic
gcaacactgtgacgtactggagggaaagtggatgggcaagcattgc

SEQ ID NO: 184 - DNA, 45 bp, Artificial Sequence
  Synthetic
gcaacactgtgacgtactggagggtgatcacctcacagctgcagg

SEQ ID NO: 185 - DNA, 48 bp, Artificial Sequence
  Synthetic
gcaacactgtgacgtactggagggtttgtttagtcaaggcatttcacc

SEQ ID NO: 186 - DNA, 45 bp, Artificial Sequence
  Synthetic
gcaacactgtgacgtactggaggcctcagcctccagagtagctgg

SEQ ID NO: 187 - DNA, 47 bp, Artificial Sequence
  Synthetic
gcaacactgtgacgtactggaggtaaaagaaaactcctccttcctgg

SEQ ID NO: 188 - DNA, 49 bp, Artificial Sequence
  Synthetic
gcaacactgtgacgtactggaggaatgtgctatgtctttaaatccatgg

SEQ ID NO: 189 - DNA, 49 bp, Artificial Sequence
  Synthetic
gcaacactgtgacgtactggaggagctggcaaatctggtaatataaaag

SEQ ID NO: 190 - DNA, 45 bp, Artificial Sequence
  Synthetic
gcaacactgtgacgtactggagggcttgaacctggaaggtggagg

SEQ ID NO: 191 - DNA, 46 bp, Artificial Sequence
  Synthetic
gcaacactgtgacgtactggagggcaggcatgctaagaccttcagc

SEQ ID NO: 192 - DNA, 47 bp, Artificial Sequence
  Synthetic
gcaacactgtgacgtactggaggcagctccatgaataactccacagg

SEQ ID NO: 193 - DNA, 45 bp, Artificial Sequence
  Synthetic
gcaacactgtgacgtactggagggcttgaacccaggaggcagagg

SEQ ID NO: 194 - DNA, 46 bp, Artificial Sequence
  Synthetic
gcaacactgtgacgtactggaggatcgaagatgccactgcaagagg

SEQ ID NO: 195 - DNA, 46 bp, Artificial Sequence
  Synthetic
gcaacactgtgacgtactggaggccaaccacacttcaggggatacc

SEQ ID NO: 196 - DNA, 46 bp, Artificial Sequence
  Synthetic
gcaacactgtgacgtactggaggcacgccagtccactgatactcac

SEQ ID NO: 197 - DNA, 45 bp, Artificial Sequence
  Synthetic
gcaacactgtgacgtactggagggggtttcaccatgttggccagg

SEQ ID NO: 198 - DNA, 45 bp, Artificial Sequence
  Synthetic
gcaacactgtgacgtactggaggcccaacaaaggctctggcctgg

SEQ ID NO: 199 - DNA, 46 bp, Artificial Sequence
  Synthetic
gcaacactgtgacgtactggaggatgacagcagaggagcttcatcc

SEQ ID NO: 200 - DNA, 46 bp, Artificial Sequence
  Synthetic
gcaacactgtgacgtactggagggcaggctacgagtaaaaggatgg

SEQ ID NO: 201 - DNA, 46 bp, Artificial Sequence
  Synthetic
gcaacactgtgacgtactggaggcgggtaaaatcttgcctccttcc

SEQ ID NO: 202 - DNA, 48 bp, Artificial Sequence
  Synthetic
gcaacactgtgacgtactggaggaaacttaaaccaatggtggatgtgg

SEQ ID NO: 203 - DNA, 46 bp, Artificial Sequence
  Synthetic
gcaacactgtgacgtactggaggagagactgaggaactgttccagc

SEQ ID NO: 204 - DNA, 47 bp, Artificial Sequence
  Synthetic
gcaacactgtgacgtactggagggaaacggtcttggatcactgatcc

SEQ ID NO: 205 - DNA, 47 bp, Artificial Sequence
  Synthetic
gcaacactgtgacgtactggaggtgcgcatgatatcttgtttcaggg

SEQ ID NO: 206 - DNA, 46 bp, Artificial Sequence
  Synthetic
gcaacactgtgacgtactggaggggcctccgtttaaactgttgtgc

SEQ ID NO: 207 - DNA, 45 bp, Artificial Sequence
  Synthetic
gcaacactgtgacgtactggagggaatgctggcccgacacagtgg

SEQ ID NO: 208 - DNA, 47 bp, Artificial Sequence
  Synthetic
gcaacactgtgacgtactggaggtcttggtatagaaaagccagctgg

SEQ ID NO: 209 - DNA, 45 bp, Artificial Sequence
  Synthetic
gcaacactgtgacgtactggagggcaaaagcccaagagcccctgg

SEQ ID NO: 210 - DNA, 46 bp, Artificial Sequence
  Synthetic
gcaacactgtgacgtactggaggttctcccaaaatgagccccaagg

SEQ ID NO: 211 - DNA, 47 bp, Artificial Sequence
  Synthetic
gcaacactgtgacgtactggagggtggtgacgtaaacaaaaggtacc

SEQ ID NO: 212 - DNA, 49 bp, Artificial Sequence
  Synthetic
gcaacactgtgacgtactggagggcaaattccatgtgaatcttattggc

SEQ ID NO: 213 - DNA, 46 bp, Artificial Sequence
  Synthetic
gcaacactgtgacgtactggaggcctgatctatggaacagtggtgg

SEQ ID NO: 214 - DNA, 48 bp, Artificial Sequence
  Synthetic
gcaacactgtgacgtactggagggttacaaacgttgcagtttgcaacg

SEQ ID NO: 215 - DNA, 46 bp, Artificial Sequence
  Synthetic
gcaacactgtgacgtactggagggaaccccgtcaacagtgatcacc

SEQ ID NO: 216 - DNA, 45 bp, Artificial Sequence
  Synthetic
gcaacactgtgacgtactggaggacaggacctcaaggcaaggagc

SEQ ID NO: 217 - DNA, 50 bp, Artificial Sequence
  Synthetic
gcaacactgtgacgtactggaggcatacctaaaatagaaatgtctatccc

SEQ ID NO: 218 - DNA, 50 bp, Artificial Sequence
  Synthetic
gcaacactgtgacgtactggagggagttgcatatatgttttataaatccc

SEQ ID NO: 219 - DNA, 47 bp, Artificial Sequence
  Synthetic
gcaacactgtgacgtactggaggtgagcccacatccataaagttagc

SEQ ID NO: 220 - DNA, 44 bp, Artificial Sequence
  Synthetic
gcaacactgtgacgtactggaggaccgcaacctttgccgcctgg

SEQ ID NO: 221 - DNA, 49 bp, Artificial Sequence
  Synthetic
gcaacactgtgacgtactggaggtaaatattttgtatggagtcaccacc

SEQ ID NO: 222 - DNA, 48 bp, Artificial Sequence
  Synthetic
gcaacactgtgacgtactggaggaaagccaggagaaaaagttatgagg

SEQ ID NO: 223 - DNA, 46 bp, Artificial Sequence
  Synthetic
gcaacactgtgacgtactggaggtcccaaagtcccaggattacagg

SEQ ID NO: 224 - DNA, 46 bp, Artificial Sequence
  Synthetic
gcaacactgtgacgtactggaggtcactatggagcatctccgatgg

SEQ ID NO: 225 - DNA, 45 bp, Artificial Sequence
  Synthetic
gcaacactgtgacgtactggaggagttccctggaagtctccgagg

SEQ ID NO: 226 - DNA, 47 bp, Artificial Sequence
  Synthetic
gcaacactgtgacgtactggaggaaaataatcacccagcccacatcc

SEQ ID NO: 227 - DNA, 48 bp, Artificial Sequence
  Synthetic
gcaacactgtgacgtactggaggacaaaactacagacacagaaagtgg

SEQ ID NO: 228 - DNA, 45 bp, Artificial Sequence
  Synthetic
gcaacactgtgacgtactggaggtttgggaggctgaggtaggtgg

SEQ ID NO: 229 - DNA, 48 bp, Artificial Sequence
  Synthetic
gcaacactgtgacgtactggaggaaagacagtgaaacatctataaggg

SEQ ID NO: 230 - DNA, 45 bp, Artificial Sequence
  Synthetic
gcaacactgtgacgtactggaggcattttgggagaccagggcagg

SEQ ID NO: 231 - DNA, 46 bp, Artificial Sequence
  Synthetic
gcaacactgtgacgtactggagggcatgggacagacacaaagcagc

SEQ ID NO: 232 - DNA, 46 bp, Artificial Sequence
  Synthetic
gcaacactgtgacgtactggagggaataacaaagagagccggctgg

SEQ ID NO: 233 - DNA, 52 bp, Artificial Sequence
  Synthetic
gcaacactgtgacgtactggaggtaaaccttttattgaaaattgtcaaatgg

SEQ ID NO: 234 - DNA, 44 bp, Artificial Sequence
  Synthetic
gcaacactgtgacgtactggaggcgcctcagcctcccaaagtgc

SEQ ID NO: 235 - DNA, 49 bp, Artificial Sequence
  Synthetic
gcaacactgtgacgtactggaggtacattagttttataggtccagtagg

SEQ ID NO: 236 - DNA, 50 bp, Artificial Sequence
  Synthetic
gcaacactgtgacgtactggagggaaggtttattcatattaaaatgtgcc

SEQ ID NO: 237 - DNA, 46 bp, Artificial Sequence
  Synthetic
gcaacactgtgacgtactggaggctggcttctgtggtttgagttgg

SEQ ID NO: 238 - DNA, 46 bp, Artificial Sequence
  Synthetic
gcaacactgtgacgtactggaggacagacctacctcctaaggatgg

SEQ ID NO: 239 - DNA, 48 bp, Artificial Sequence
  Synthetic
gcaacactgtgacgtactggagggctagcttttgtgtgtaagaatggg

SEQ ID NO: 240 - DNA, 47 bp, Artificial Sequence
  Synthetic
gcaacactgtgacgtactggaggggcctactcacacaatagaatacc

SEQ ID NO: 241 - DNA, 45 bp, Artificial Sequence
  Synthetic
gcaacactgtgacgtactggagggcaccattgcactccagcctgg

SEQ ID NO: 242 - DNA, 49 bp, Artificial Sequence
  Synthetic
gcaacactgtgacgtactggagggaaattaggataaaggttgtcacagc

SEQ ID NO: 243 - DNA, 48 bp, Artificial Sequence
  Synthetic
gcaacactgtgacgtactggaggcagaagtgttcaaggtgaaactgtc

SEQ ID NO: 244 - DNA, 49 bp, Artificial Sequence
  Synthetic
gcaacactgtgacgtactggaggctgaatcatgaaatgttctactctgc

SEQ ID NO: 245 - DNA, 46 bp, Artificial Sequence
  Synthetic
gcaacactgtgacgtactggaggtgtcaacttgactgggccatacg

SEQ ID NO: 246 - DNA, 47 bp, Artificial Sequence
  Synthetic
gcaacactgtgacgtactggaggctcccgtatagttgggattatagg

SEQ ID NO: 247 - DNA, 48 bp, Artificial Sequence
  Synthetic
gcaacactgtgacgtactggagggcttggagttccttgaaattcttgg

SEQ ID NO: 248 - DNA, 46 bp, Artificial Sequence
  Synthetic
gcaacactgtgacgtactggaggcctggtggctccagttttctacc

SEQ ID NO: 249 - DNA, 46 bp, Artificial Sequence
  Synthetic
gcaacactgtgacgtactggaggaactcctgacctcatgatccacc

SEQ ID NO: 250 - DNA, 45 bp, Artificial Sequence
  Synthetic
gcaacactgtgacgtactggagggctgggattacaggcatgagcc

SEQ ID NO: 251 - DNA, 48 bp, Artificial Sequence
  Synthetic
gcaacactgtgacgtactggaggttctcctttatccttggtgacattc

SEQ ID NO: 252 - DNA, 46 bp, Artificial Sequence; identical to SEQ ID NO: 172
  Synthetic

SEQ ID NO: 253 - DNA, 46 bp, Artificial Sequence
  Synthetic
gcaacactgtgacgtactggagggtcataagtcagggaccatctgc

SEQ ID NO: 254 - DNA, 48 bp, Artificial Sequence
  Synthetic
gcaacactgtgacgtactggaggctgtttcattgatttccagactggc

SEQ ID NO: 255 - DNA, 45 bp, Artificial Sequence
  Synthetic
gcaacactgtgacgtactggagggcaatctcggctcactgcaagc

SEQ ID NO: 256 - DNA, 48 bp, Artificial Sequence
  Synthetic
gcaacactgtgacgtactggagggaagaagtgactatatcagatctgg

SEQ ID NO: 257 - DNA, 45 bp, Artificial Sequence
  Synthetic
gcaacactgtgacgtactggaggttcaccatgttggccaggctgg

SEQ ID NO: 258 - DNA, 47 bp, Artificial Sequence
  Synthetic
gcaacactgtgacgtactggaggcatcactgaagatgacaactgagc

SEQ ID NO: 259 - DNA, 44 bp, Artificial Sequence
  Synthetic
gcaacactgtgacgtactggagggtccagcctgggcgatagagc

SEQ ID NO: 260 - DNA, 47 bp, Artificial Sequence
  Synthetic
gcaacactgtgacgtactggagggaggaaagtctttgaagaggaacc

SEQ ID NO: 261 - DNA, 46 bp, Artificial Sequence
  Synthetic
gcaacactgtgacgtactggaggggtacactcaccagcagttttgc

SEQ ID NO: 262 - DNA, 48 bp, Artificial Sequence
  Synthetic
gcaacactgtgacgtactggagggagcaactggtgtgaatacatatgg

SEQ ID NO: 263 - DNA, 46 bp, Artificial Sequence
  Synthetic
gcaacactgtgacgtactggaggcaatacctggcaccacatacacc

SEQ ID NO: 264 - DNA, 45 bp, Artificial Sequence
  Synthetic
gcaacactgtgacgtactggagggggactacaggcatgtgccacc

SEQ ID NO: 265 - DNA, 45 bp, Artificial Sequence
  Synthetic
gcaacactgtgacgtactggaggcggtggctcacgcgtgtaatcc

SEQ ID NO: 266 - DNA, 49 bp, Artificial Sequence
  Synthetic
gcaacactgtgacgtactggaggcaactgttaaatctctcatggaaacc

SEQ ID NO: 267 - DNA, 46 bp, Artificial Sequence
  Synthetic
gcaacactgtgacgtactggagggacaaaggattagaaatgcaccc

SEQ ID NO: 268 - DNA, 49 bp, Artificial Sequence
  Synthetic
gcaacactgtgacgtactggaggggaaatgttctaaaactggattgtgg

SEQ ID NO: 269 - DNA, 48 bp, Artificial Sequence
  Synthetic
gcaacactgtgacgtactggaggaataataatagccaggtgtggtagc

SEQ ID NO: 270 - DNA, 46 bp, Artificial Sequence
  Synthetic
gcaacactgtgacgtactggaggctggaacactcacacattgctgg

SEQ ID NO: 271 - DNA, 45 bp, Artificial Sequence
  Synthetic
gcaacactgtgacgtactggaggctgggtgacagagcgagactcc

SEQ ID NO: 272 - DNA, 48 bp, Artificial Sequence
  Synthetic
gcaacactgtgacgtactggaggcccaaatcatccccgtgaaacatgc

SEQ ID NO: 273 - DNA, 45 bp, Artificial Sequence
  Synthetic
gcaacactgtgacgtactggagggaccctgcaatcccaacactgg

SEQ ID NO: 274 - DNA, 46 bp, Artificial Sequence
  Synthetic
gcaacactgtgacgtactggaggctctcaggccttcaaactacacc

SEQ ID NO: 275 - DNA, 45 bp, Artificial Sequence
  Synthetic
gcaacactgtgacgtactggaggcaggaaagggctcgctcagtgg

SEQ ID NO: 276 - DNA, 46 bp, Artificial Sequence
  Synthetic
gcaacactgtgacgtactggaggatctgcaaaagcagcagagcagg

SEQ ID NO: 277 - DNA, 48 bp, Artificial Sequence
  Synthetic
gcaacactgtgacgtactggagggtacccatgacagacaagttttagg

SEQ ID NO: 278 - DNA, 47 bp, Artificial Sequence
  Synthetic
gcaacactgtgacgtactggaggcttatcccctactgtctcctttgg

SEQ ID NO: 279 - DNA, 45 bp, Artificial Sequence
  Synthetic
gcaacactgtgacgtactggaggggatggtctcgatctcctgacc

SEQ ID NO: 280 - DNA, 47 bp, Artificial Sequence
  Synthetic
gcaacactgtgacgtactggaggaggttagagaccttcctctaatgc

SEQ ID NO: 281 - DNA, 45 bp, Artificial Sequence
  Synthetic
gcaacactgtgacgtactggaggagctgggattacaggtgcctgc

SEQ ID NO: 282 - DNA, 44 bp, Artificial Sequence
  Synthetic
gcaacactgtgacgtactggagggctgaggcaggttggggctgc

SEQ ID NO: 283 - DNA, 48 bp, Artificial Sequence
  Synthetic
gcaacactgtgacgtactggaggacatttaacgtctcctaacttctcc

SEQ ID NO: 284 - DNA, 46 bp, Artificial Sequence
  Synthetic
gcaacactgtgacgtactggagggtgctgcgattacaggtgtgagc

SEQ ID NO: 285 - DNA, 49 bp, Artificial Sequence
  Synthetic
gcaacactgtgacgtactggaggtatgacagcagtattatactatcacc

SEQ ID NO: 286 - DNA, 46 bp, Artificial Sequence
  Synthetic
gcaacactgtgacgtactggaggctggggaccaaatctgaactgcc

SEQ ID NO: 287 - DNA, 49 bp, Artificial Sequence
  Synthetic
gcaacactgtgacgtactggagggtagctattgttatttccaaaagagg

SEQ ID NO: 288 - DNA, 44 bp, Artificial Sequence
  Synthetic
gcaacactgtgacgtactggagggcttgggaccccaggacaagg

SEQ ID NO: 289 - DNA, 45 bp, Artificial Sequence
  Synthetic
gcaacactgtgacgtactggaggcctggccaacatggggaaatcc

SEQ ID NO: 290 - DNA, 46 bp, Artificial Sequence
  Synthetic
gcaacactgtgacgtactggaggaattgcttgaacctgggaggtgg

SEQ ID NO: 291 - DNA, 47 bp, Artificial Sequence
  Synthetic
gcaacactgtgacgtactggagggcctaagacccaaaagctattagc

SEQ ID NO: 292 - DNA, 50 bp, Artificial Sequence
  Synthetic
gcaacactgtgacgtactggaggcatattaaagggccatattcaaattgg

SEQ ID NO: 293 - DNA, 48 bp, Artificial Sequence
  Synthetic
gcaacactgtgacgtactggaggggatgtaaccagtgtatatcacagg

SEQ ID NO: 294 - DNA, 48 bp, Artificial Sequence
  Synthetic
gcaacactgtgacgtactggaggggaagtttagtccacatcttctagc

SEQ ID NO: 295 - DNA, 45 bp, Artificial Sequence
  Synthetic
gcaacactgtgacgtactggagggcacccacaggacaaccacacg

SEQ ID NO: 296 - DNA, 45 bp, Artificial Sequence
  Synthetic
gcaacactgtgacgtactggagggggacgcgcctgttaacaaagg

SEQ ID NO: 297 - DNA, 42 bp, Artificial Sequence
  Synthetic
gcaacactgtgacgtactggagggggctgggggccacgctcc

SEQ ID NO: 298 - DNA, 45 bp, Artificial Sequence
  Synthetic
gcaacactgtgacgtactggaggcgcaaaagtgaagccctcctgg

SEQ ID NO: 299 - DNA, 49 bp, Artificial Sequence
  Synthetic
gcaacactgtgacgtactggagggaaatcctacttgatctaaagtgagc

SEQ ID NO: 300 - DNA, 49 bp, Artificial Sequence
  Synthetic
gcaacactgtgacgtactggaggtttgagcaacttggaaaaaataagcg

SEQ ID NO: 301 - DNA, 48 bp, Artificial Sequence
  Synthetic
gcaacactgtgacgtactggaggttcccaaaagacaaatagcacttcc

SEQ ID NO: 302 - DNA, 49 bp, Artificial Sequence
  Synthetic
gcaacactgtgacgtactggaggccattttgaaaatcacagtgaattcc

SEQ ID NO: 303 - DNA, 49 bp, Artificial Sequence
  Synthetic
gcaacactgtgacgtactggagggaaaagaaaaccctgaattcaaaagg

SEQ ID NO: 304 - DNA, 49 bp, Artificial Sequence
  Synthetic
gcaacactgtgacgtactggaggtgctgaaaagaagcatttaaaagtgg

SEQ ID NO: 305 - DNA, 47 bp, Artificial Sequence
  Synthetic
gcaacactgtgacgtactggaggctcttaccagtttcagagctttcc

SEQ ID NO: 306 - DNA, 48 bp, Artificial Sequence
  Synthetic
gcaacactgtgacgtactggaggttttcagccaaaaatcaaggacagg

SEQ ID NO: 307 - DNA, 46 bp, Artificial Sequence
  Synthetic
gcaacactgtgacgtactggaggcttgagcccaggagtttgagacc

SEQ ID NO: 308 - DNA, 46 bp, Artificial Sequence
  Synthetic
gcaacactgtgacgtactggaggcgcctgtagtaccctctactagg

SEQ ID NO: 309 - DNA, 49 bp, Artificial Sequence
  Synthetic
gcaacactgtgacgtactggaggggtaaagaaagaaggatttgaaaacc

SEQ ID NO: 310 - DNA, 50 bp, Artificial Sequence
  Synthetic
gcaacactgtgacgtactggaggtaagagtaatgaggttaaagtttatgc

SEQ ID NO: 311 - DNA, 49 bp, Artificial Sequence
  Synthetic
gcaacactgtgacgtactggaggcatttttattgtcacaggccatttgc

SEQ ID NO: 312 - DNA, 46 bp, Artificial Sequence
  Synthetic
gcaacactgtgacgtactggagggccacgccttctcttctgccacc

SEQ ID NO: 313 - DNA, 45 bp, Artificial Sequence
  Synthetic
gcaacactgtgacgtactggaggtgcctctcctgactgcactgtg

SEQ ID NO: 314 - DNA, 45 bp, Artificial Sequence
  Synthetic
gcaacactgtgacgtactggaggccatgctctaccacgcccttgg

SEQ ID NO: 315 - DNA, 45 bp, Artificial Sequence
  Synthetic
gcaacactgtgacgtactggaggcattcaggctggagtgcggtgg

SEQ ID NO: 316 - DNA, 50 bp, Artificial Sequence
  Synthetic
gcaacactgtgacgtactggaggcttaaaaattgtctggctaagacattg

SEQ ID NO: 317 - DNA, 45 bp, Artificial Sequence
  Synthetic
gcaacactgtgacgtactggaggttgctcttgttgcccgggttgg

SEQ ID NO: 318 - DNA, 49 bp, Artificial Sequence
  Synthetic
gcaacactgtgacgtactggagggagcttagaggaaaagtattatttcc

SEQ ID NO: 319 - DNA, 44 bp, Artificial Sequence
  Synthetic
gcaacactgtgacgtactggaggtggtgctgtgccagacgctgg

SEQ ID NO: 320 - DNA, 48 bp, Artificial Sequence
  Synthetic
gcaacactgtgacgtactggaggcagatctttttggctattgtcttgg

SEQ ID NO: 321 - DNA, 45 bp, Artificial Sequence
  Synthetic
gcaacactgtgacgtactggagggaaggaaagggcctcccactgc

SEQ ID NO: 322 - DNA, 46 bp, Artificial Sequence
  Synthetic
gcaacactgtgacgtactggaggcatgaaaaagcatgctggggagg

SEQ ID NO: 323 - DNA, 51 bp, Artificial Sequence
  Synthetic
gcaacactgtgacgtactggaggcaaacataaaaaagctttaatagaagcc

SEQ ID NO: 324 - DNA, 49 bp, Artificial Sequence
  Synthetic
gcaacactgtgacgtactggaggtcccaactatgaaaaaatagaagacg

SEQ ID NO: 325 - DNA, 46 bp, Artificial Sequence
  Synthetic
gcaacactgtgacgtactggaggcacaaattagccgggcatggtgg

SEQ ID NO: 326 - DNA, 49 bp, Artificial Sequence
  Synthetic
gcaacactgtgacgtactggaggcttcctttactgagtctttctaaagc

SEQ ID NO: 327 - DNA, 48 bp, Artificial Sequence
  Synthetic
gcaacactgtgacgtactggaggtgtcctttgaaatgtaggtatgtgg

SEQ ID NO: 328 - DNA, 47 bp, Artificial Sequence
  Synthetic
gcaacactgtgacgtactggaggggatcttgcaatactgacatctcc

SEQ ID NO: 329 - DNA, 49 bp, Artificial Sequence
  Synthetic
gcaacactgtgacgtactggaggatttgaaaagaactgaaggatctacc

SEQ ID NO: 330 - DNA, 46 bp, Artificial Sequence
  Synthetic
gcaacactgtgacgtactggagggtgagctgagatctcgtctctgc

SEQ ID NO: 331 - DNA, 51 bp, Artificial Sequence
  Synthetic
gcaacactgtgacgtactggaggtttgtctgaaacagattctaaaagttgg

SEQ ID NO: 332 - DNA, 44 bp, Artificial Sequence
  Synthetic
gcaacactgtgacgtactggagggcaggtgcctgtagtcccagc

SEQ ID NO: 333 - DNA, 50 bp, Artificial Sequence
  Synthetic
gcaacactgtgacgtactggagggtttgagcttctaaaattcatggattc

SEQ ID NO: 334 - DNA, 46 bp, Artificial Sequence
  Synthetic
gcaacactgtgacgtactggagggtggtaggtcaaaccgcaattcc

SEQ ID NO: 335 - DNA, 48 bp, Artificial Sequence
  Synthetic
gcaacactgtgacgtactggaggaccaaatcagacatatcagctttgg

SEQ ID NO: 336 - DNA, 47 bp, Artificial Sequence
  Synthetic
gcaacactgtgacgtactggaggcacagaacggatcctcaataaagg

SEQ ID NO: 337 - DNA, 48 bp, Artificial Sequence
  Synthetic
gcaacactgtgacgtactggagggttaactcctcccttctctttatgg

SEQ ID NO: 338 - DNA, 41 bp, Artificial Sequence
  Synthetic
gtctcaagtgagctacctggtggcaggaggagccccagagc

SEQ ID NO: 339 - DNA, 43 bp, Artificial Sequence
  Synthetic
gtctcaagtgagctacctggtggtcctggggatggttggatgc

SEQ ID NO: 340 - DNA, 46 bp, Artificial Sequence
  Synthetic
gtctcaagtgagctacctggtggtgaccccacagagtttacacagc

SEQ ID NO: 341 - DNA, 42 bp, Artificial Sequence
  Synthetic
gtctcaagtgagctacctggtggagtcagggcaggctctgcc

SEQ ID NO: 342 - DNA, 46 bp, Artificial Sequence
  Synthetic
gtctcaagtgagctacctggtggtattttggccccatccagaaagc

SEQ ID NO: 343 - DNA, 46 bp, Artificial Sequence
  Synthetic
gtctcaagtgagctacctggtggcacccagagtacagctttgttcc

SEQ ID NO: 344 - DNA, 40 bp, Artificial Sequence
  Synthetic
gactgcgaggatcaggtctccgaggagccccagagcctgc

SEQ ID NO: 345 - DNA, 43 bp, Artificial Sequence
  Synthetic
gactgcgaggatcaggtctcctggggatggttggatgcttacc

SEQ ID NO: 346 - DNA, 44 bp, Artificial Sequence
  Synthetic
gactgcgaggatcaggtctcccccacagagtttacacagcttgc

SEQ ID NO: 347 - DNA, 40 bp, Artificial Sequence
  Synthetic
gactgcgaggatcaggtctcccaggctctgcccactcacc

SEQ ID NO: 348 - DNA, 42 bp, Artificial Sequence
  Synthetic
gactgcgaggatcaggtctccccatccagaaagcccaaagcc

SEQ ID NO: 349 - DNA, 47 bp, Artificial Sequence
  Synthetic
gactgcgaggatcaggtctccccagagtacagctttgttcctcattc

SEQ ID NO: 350 - DNA, 36 bp, Artificial Sequence
  Synthetic
cggacagcccgtcagacgccagagcctgcaagctgc

SEQ ID NO: 351 - DNA, 41 bp, Artificial Sequence
  Synthetic
cggacagcccgtcagacgtggttggatgcttacccaaaacc

SEQ ID NO: 352 - DNA, 42 bp, Artificial Sequence
  Synthetic
cggacagcccgtcagacgcagagtttacacagcttgccttgg

SEQ ID NO: 353 - DNA, 38 bp, Artificial Sequence
  Synthetic
cggacagcccgtcagacgctctgcccactcacccctgc

SEQ ID NO: 354 - DNA, 40 bp, Artificial Sequence
  Synthetic
cggacagcccgtcagacgcagaaagcccaaagccaacagg

SEQ ID NO: 355 - DNA, 44 bp, Artificial Sequence
  Synthetic
cggacagcccgtcagacgagtacagctttgttcctcattctgac

SEQ ID NO: 356 - DNA, 39 bp, Artificial Sequence
  Synthetic
ggaatagggaagaaacgcaggccctgcaagctgccgtgc

SEQ ID NO: 357 - DNA, 45 bp, Artificial Sequence
  Synthetic
ggaatagggaagaaacgcaggcgcttacccaaaaccatacagtgg

SEQ ID NO: 358 - DNA, 46 bp, Artificial Sequence
  Synthetic
ggaatagggaagaaacgcaggcacagcttgccttgggtgttataac

SEQ ID NO: 359 - DNA, 41 bp, Artificial Sequence
  Synthetic
ggaatagggaagaaacgcaggcactcacccctgcaggcacc

SEQ ID NO: 360 - DNA, 44 bp, Artificial Sequence
  Synthetic
ggaatagggaagaaacgcaggcagcccaaagccaacaggagtcc

SEQ ID NO: 361 - DNA, 46 bp, Artificial Sequence
  Synthetic
ggaatagggaagaaacgcaggcctttgttcctcattctgactgagc

SEQ ID NO: 362 - DNA, 21 bp, Artificial Sequence
  Synthetic
gactgcgaggatcaggtctcc

SEQ ID NO: 363 - DNA, 18 bp, Artificial Sequence
  Synthetic
cggacagcccgtcagacg

SEQ ID NO: 364 - DNA, 22 bp, Artificial Sequence
  Synthetic
ggaatagggaagaaacgcaggc

SEQ ID NO: 365 - DNA, 42 bp, Artificial Sequence
  Synthetic
gcagtacaaacaacgcacagcgctgccaccctccacagtccc

SEQ ID NO: 366 - DNA, 40 bp, Artificial Sequence
  Synthetic
gcagtacaaacaacgcacagcggccaagaccatgcatgcg

SEQ ID NO: 367 - DNA, 43 bp, Artificial Sequence
  Synthetic
gcagtacaaacaacgcacagcgcccagggacaaagagactccc

SEQ ID NO: 368 - DNA, 48 bp, Artificial Sequence
  Synthetic
gcagtacaaacaacgcacagcgcaggaagcagacagtcttctagttcc

SEQ ID NO: 369 - DNA, 45 bp, Artificial Sequence
  Synthetic
gcagtacaaacaacgcacagcgtgcctgtaatcccaacactttgg

SEQ ID NO: 370 - DNA, 41 bp, Artificial Sequence
  Synthetic
gcagtacaaacaacgcacagcgtccctctggccaggatggg

SEQ ID NO: 371 - DNA, 44 bp, Artificial Sequence
  Synthetic
gcagtacaaacaacgcacagcgatggggaatgggagtaggaagc

SEQ ID NO: 372 - DNA, 44 bp, Artificial Sequence
  Synthetic
gcagtacaaacaacgcacagcgcagatcagttctcccctccagc

SEQ ID NO: 373 - DNA, 48 bp, Artificial Sequence
  Synthetic
gcagtacaaacaacgcacagcgacaaaaaagaaacatgctcagagagg

SEQ ID NO: 374 - DNA, 44 bp, Artificial Sequence
  Synthetic
gcagtacaaacaacgcacagcgtggtggcatgcatctgtagtcc

SEQ ID NO: 375 - DNA, 48 bp, Artificial Sequence
  Synthetic
gcagtacaaacaacgcacagcgaggtgctctatagatgttagcatccc

SEQ ID NO: 376 - DNA, 44 bp, Artificial Sequence
  Synthetic
gcagtacaaacaacgcacagcgccaggacaggatggagatctgg

SEQ ID NO: 377 - DNA, 46 bp, Artificial Sequence
  Synthetic
gcagtacaaacaacgcacagcgagggaacctgtgcattatccttgc

SEQ ID NO: 378 - DNA, 47 bp, Artificial Sequence
  Synthetic
gcagtacaaacaacgcacagcgcagaagtcttgctttaaggaggagg

SEQ ID NO: 379 - DNA, 44 bp, Artificial Sequence
  Synthetic
gcagtacaaacaacgcacagcggggtacgtgaaactcaccaagg

SEQ ID NO: 380 - DNA, 42 bp, Artificial Sequence
  Synthetic
gcagtacaaacaacgcacagcgcagagtgtggcaagcaaggg

SEQ ID NO: 381 - DNA, 50 bp, Artificial Sequence
  Synthetic
gcagtacaaacaacgcacagcgaacattttaaaggtacaaataacgtggg

SEQ ID NO: 382 - DNA, 44 bp, Artificial Sequence
  Synthetic
gcagtacaaacaacgcacagcgtagggagcaacagccattaagc

SEQ ID NO: 383 - DNA, 42 bp, Artificial Sequence
  Synthetic
gcagtacaaacaacgcacagcgggtgcactgtccagctctgg

SEQ ID NO: 384 - DNA, 43 bp, Artificial Sequence
  Synthetic
gcagtacaaacaacgcacagcgactctcgctgaactcgcctgg

SEQ ID NO: 385 - DNA, 42 bp, Artificial Sequence
  Synthetic
gcagtacaaacaacgcacagcgctcggtctctggtggtacgc

SEQ ID NO: 386 - DNA, 41 bp, Artificial Sequence
  Synthetic
gcagtacaaacaacgcacagcggcaagaggtccgagctggg

SEQ ID NO: 387 - DNA, 48 bp, Artificial Sequence
  Synthetic
gcagtacaaacaacgcacagcgggaagaagtgaaacaagagatgaagg

SEQ ID NO: 388 - DNA, 45 bp, Artificial Sequence
  Synthetic
gcagtacaaacaacgcacagcgcccagagaacaaaccggattagg

SEQ ID NO: 389 - DNA, 45 bp, Artificial Sequence
  Synthetic
gcagtacaaacaacgcacagcgcccttcaaccttctccaatctgc

SEQ ID NO: 390 - DNA, 43 bp, Artificial Sequence
  Synthetic
gcagtacaaacaacgcacagcgcccatgtccagtggtttaggg

SEQ ID NO: 391 - DNA, 44 bp, Artificial Sequence
  Synthetic
gcagtacaaacaacgcacagcggagattggtgggagacagatgg

SEQ ID NO: 392 - DNA, 45 bp, Artificial Sequence
  Synthetic
gcagtacaaacaacgcacagcgcttctcagctcaaagttccagcg

SEQ ID NO: 393 - DNA, 44 bp, Artificial Sequence
  Synthetic
gcagtacaaacaacgcacagcggaatgggagagatgaccagagg

SEQ ID NO: 394 - DNA, 42 bp, Artificial Sequence
  Synthetic
gcagtacaaacaacgcacagcgaagggcaagggggtatgtgg

SEQ ID NO: 395 - DNA, 43 bp, Artificial Sequence
  Synthetic
gcagtacaaacaacgcacagcgggaaggaagcatgggaacacc

SEQ ID NO: 396 - DNA, 45 bp, Artificial Sequence
  Synthetic
gcagtacaaacaacgcacagcgccatcaatgctctgtctgtctgg

SEQ ID NO: 397 - DNA, 41 bp, Artificial Sequence
  Synthetic
gcagtacaaacaacgcacagcggtgccgtgactgtgcttgg

SEQ ID NO: 398 - DNA, 45 bp, Artificial Sequence
  Synthetic
gcagtacaaacaacgcacagcgacatcccattgacctcatcaagc